SEQUENCE LISTING (24 sequences)

SEQ ID NO: 1 - protein, 19 aa, Artificial Sequence
  Synthetic peptide
DRKFLMECRNSPVAKTPPK

SEQ ID NO: 2 - protein, 13 aa, Artificial Sequence
  Synthetic peptide
DKRAGGEESQFEM

SEQ ID NO: 3 - protein, 9 aa, Artificial Sequence
  Synthetic peptide
SAGTATQMR

SEQ ID NO: 4 - protein, 11 aa, Artificial Sequence
  Synthetic peptide
SAPPNLWAAQR

SEQ ID NO: 5 - protein, 14 aa, Artificial Sequence
  Synthetic peptide
RVESPVLPPVLVPR

SEQ ID NO: 6 - protein, 15 aa, Artificial Sequence
  Synthetic peptide
HSEYNPQHSLLAQFR

SEQ ID NO: 7 - protein, 17 aa, Artificial Sequence
  Synthetic peptide
CHVSTHQHNYAAPPSTR

SEQ ID NO: 8 - protein, 13 aa, Artificial Sequence
  Synthetic peptide
FELLPTPPLSPSR

SEQ ID NO: 9 - protein, 14 aa, Artificial Sequence
  Synthetic peptide
FELLPTPPLSPSRR

SEQ ID NO: 10 - protein, 7 aa, Artificial Sequence
  Synthetic peptide
VKLDSVR

SEQ ID NO: 11 - protein, 20 aa, Artificial Sequence
  Synthetic peptide
MYGISLCQAILDETKGDYEK

SEQ ID NO: 12 - protein, 30 aa, Artificial Sequence
  Synthetic peptide
AAVSHWQQQSYLDSGIHSGATTTAPSLSGK

SEQ ID NO: 13 - protein, 15 aa, Artificial Sequence
  Synthetic peptide
TSMGGTQQQFVEGVR

SEQ ID NO: 14 - protein, 12 aa, Artificial Sequence
  Synthetic peptide
DIPGLTDTTVPR

SEQ ID NO: 15 - protein, 13 aa, Artificial Sequence
  Synthetic peptide
DIPGLTDTTVPRR

SEQ ID NO: 16 - protein, 8 aa, Artificial Sequence
  Synthetic peptide
WPGSPTSR

SEQ ID NO: 17 - protein, 8 aa, Artificial Sequence
  Synthetic peptide
HNLSLHSR

SEQ ID NO: 18 - protein, 12 aa, Artificial Sequence
  Synthetic peptide
AVSMDNSNKYTK

SEQ ID NO: 19 - protein, 30 aa, Artificial Sequence
  Synthetic peptide
TPPRDLPTIPGVTSPSSDEPPMEASQSHLR

SEQ ID NO: 20 - protein, 12 aa, Artificial Sequence
  Synthetic peptide
SVTCTYSPALNK

SEQ ID NO: 21 - protein, 13 aa, Artificial Sequence
  Synthetic peptide
ALPNNTSSSPQPK

SEQ ID NO: 22 - protein, 15 aa, Artificial Sequence
  Synthetic peptide
CSDSDGLAPPQHLIR

SEQ ID NO: 23 - protein, 14 aa, Artificial Sequence
  Synthetic peptide
RALPNNTSSSPQPK

SEQ ID NO: 24 - protein, 7 aa, Artificial Sequence
  Synthetic peptide
RPRAATF

(19) United States
(12) Patent Application Publication
(10) Pub. No.: US 2011/0281289 A1
(43) Pub. Date: Nov. 17, 2011

(54) QUANTIFICATION OF ENZYME ACTIVITY BY MASS SPECTROMETRY USING IMMOBILIZED SUBSTRATES

(75) Inventors: Pedro Cutillas, Surrey (GB); Bart Vanhaesebroeck, London (GB)

(73) Assignee: LUDWIG INSTITUTE FOR CANCER RESEARCH LTD., New York, NY (US)

(21) Appl. No.: 13/003,731

(22) PCT Filed: Jul. 13, 2009
(86) PCT No.: PCT/US09/50407

§ 371 Date: Jul. 28, 2011

Related U.S. Application Data
(60) Provisional application No. 61/080,014, filed on Jul. 11, 2008.

(51) Int. Cl. C12Q 1/48; C12Q 1/00

(52) U.S. Cl. 435/15; 435/4

(57) ABSTRACT

The disclosure relates to methods of analyzing the enzymatic activity of enzymes in samples containing a plurality of enzymes, using mass spectrometry. Immobilized substrates are employed. Purified enzymes and enzymes from crude cell lysates can be analyzed using the disclosed methods.

### CROSS REFERENCE TO RELATED APPLICATIONS

This application claims the benefit of U.S. Provisional Application No. 61/080,014 filed Jul. 11, 2008, which is incorporated by reference in its entirety herein.

### BACKGROUND

1. Field of the Invention

The present invention relates to material and methods for the quantification of enzyme activity in a sample. In particular, the present invention relates to methods of quantifying enzyme activity using spectroscopy such as mass spectroscopy. The information obtained is valuable for biological research; pharmaceutical research and development; medical diagnosis, prophylaxis, and therapy; forensics; and many other practical applications.

2. Related Technology

Mutations that directly or indirectly activate signaling pathways downstream of growth factor receptors confer growth advantage to cancer cells. The fluxes of these signaling pathways are controlled by the activities of several enzymes, of which lipid and protein kinases play prominent roles. Thus protein kinases control fundamental cell physiological processes and are also implicated in numerous pathological processes including cancer. There are at least 518 protein kinases in the human genome. Several of these are validated drug targets for the treatment of diverse forms of cancer (e.g., receptor tyrosine kinase, RTK), and others are the target of inhibitors that show promise in pre-clinical studies and in early phase clinical trials (see, e.g., Weisberg, et al, Nat Rev Cancer 7:345-56 (2007); Quintas-Cardama et al. Nat Rev Drug Discov 6:834-48 (2007); Mackay et al. Nat Rev Cancer 7:554-62 (2007); Faivre, et al., Nat Rev Drug Discov 6:734-45 (2007); Wilhelm, et al, Nat Rev Drug Discov 5:835-44 (2006); and Faivre, et al., Nat Rev Drug Discov 5:671-88 (2006)). Protein kinases are also the target of other drugs developed to treat autoimmune conditions and allergy.

Understanding the role of protein kinases in disease requires methods that can be used to detect and quantify their activities. This is also important for the success of therapies that target this enzyme group. As an example, cancers are heterogeneous biological entities, which in practical terms mean that not all cancer cells respond to the inhibition of a signaling pathway to the same extent, a fact that complicates therapy. The ability to assess how active signaling pathways are in tumor cells could be used to predict sensitivity to therapies that target pathways members. This paradigm has proved to be of extreme importance for the success of cancer therapies that target RTKs (Krause et al., Cancer Metastasis Rev, (2008)) and further progress in the design of therapies that target signaling modules is hampered by the difficulty in finding ideal biomarkers of pathway activation (Sawyers, Nature 452:548-52 (2008)). Therefore, methods to assess the activity of signaling pathways driven by protein kinases would be useful to provide a basic understanding of oncogenic signaling (i.e., as a readout of biological and pharmacological experiments), for the success of clinical trials (to enroll the most appropriate patient group in the study), and to individualize therapies based on cell signaling inhibitors.

The activity of protein kinases can be affected by genetic mutations, and consequently there is a strong interest in detecting the mutations in this enzyme group that may drive the onset and progression of cancer (Greenman et al., Nature 446:153-8 (2007)). However, it may be difficult to correlate how specific genetic mutations may affect enzyme, and hence pathway, activity (Haber et al., Nature 446:145-6 (2007)). Indeed, enzymatic activity can be modulated by a large array of molecular phenomena in addition to mutations on their genetic sequence, including enzyme gene expression (i.e., their amounts in cells), protein-protein interactions and other allosteric modulators, miRNAs, epigenetic modifications, protein posttranslational modifications, and activities of upstream pathway members (some of which may remain to be discovered or may not be obvious a priori). Therefore, methods for direct measurement of enzymatic (e.g., kinase) activities would be valuable as a readout for biological experiments, for drug development and monitoring of efficacy in patients, and as a source of predictive biomarkers.

Approaches for the unbiased detection of enzymatic activities have been reported. It is possible to use chemical probes to covalently link reactive amino acids in enzyme active sites (Blethrow et al., Proc Natl Acad Sci USA 105:1442-7 (2008)) or on the substrates (Barglow et al., Nat Methods 4:822-7 (2007)). Proteins linked to the probes are affinity purified and their identities determined by mass spectrometry. This approach can detect activities and substrates, but it requires a large number of cells and the information provided is only qualitative. As a more quantitative approach, quantification of phosphorylation sites on proteins known to be substrates of specific kinases serves as a measure of kinase activity. When performed using mass spectrometry as the readout, this approach allows quantifying hundreds to thousands of phosphorylation sites in a single experiment. As an example, using metabolic labeling with stable isotopes (the SILAC approach) it was possible to quantify >2000 phosphorylation sites that showed altered levels of expression upon treatment of HeLa cells with EGF (Olsen et al., Cell 127:635-48 (2006)). However, since cells need to be metabolically active to incorporate labeled amino acids, this approach cannot be used as a general tool to quantify cell signaling in primary tissues, and its low throughput puts limits to its usefulness. The use of isotope labeled internal standard peptides to measure phosphorylated peptides could be an alternative (Gerber, et al., Proc Natl Acad Sci USA 100:6940-5 (2003)) but direct quantification of phosphorylation sites also considers the action of cellular phosphatases or other enzymes, which can complicate the interpretation of certain experiments. In addition, despite improvements in the sensitivity and dynamic range of modern mass spectrometers, quantification of phosphorylation by mass spectrometry still requires a large number of cells and extensive sample fractionation, thus making the approach unsuitable to study signaling in human primary tissues and in clinical specimens.

Previous reports of measuring kinase activity by mass spectrometry have provided a sensitive and specific means to quantify the PI3K/Akt signaling pathway using a small peptide substrate (see, e.g., Cutillas et al., Proc Natl Acad Sci USA 103:8959-64 (2006) and WO 07/127,767). This assay is based on the use of a synthetic peptide that serves as a substrate for kinases downstream PI3K. Since the assay is based in an enzymatic reaction, it allows amplifying the signal of the target kinase(s) and this amplification allows measuring signaling with great sensitivity. Having mass spectrometry as the readout makes the approach precise, accurate and with large dynamic ranges (determined by the type of mass spectrometer used but 100-100000 fold dynamic ranges are routine). For this approach to be of general use to quantify other pathways, it is important to identify small peptide substrates that serve as specific readouts of different pathways. This may not always be possible because specificity of protein kinases to their different substrates may sometimes require long-range molecular interactions (see, e.g., Biondi et al., Biochem J 372:1-13 (2003)), a type of intermolecular contact that cannot be mimicked with short substrate peptides. Thus, a need exists for methods for detecting activity of enzymes of interest which is quantitative and can be done using larger substrates than short substrate peptides.

### SUMMARY

The present disclosure addresses the need for materials and methods for analyzing enzyme activities of samples to yield data that may be compared across samples.

One aspect of the invention is a method for detecting the activity of an enzyme in a sample that contains one or more enzymes. For example, in one variation, the method comprises: incubating the sample with a first particle to start a first enzymatic reaction, wherein the first particle has, attached to its surface, a first substrate for a first enzyme that is known or suspected of being present in the sample, and the incubating is under conditions effective to permit a first enzymatic reaction involving the first enzyme and the first substrate to produce a first product, the first product being attached to the surface of the first particle; isolating the first particle from the mixture, wherein the isolated first particle includes, attached to its surface, first product and unreacted first substrate; contacting the first product and the unreacted first substrate with a cleavage agent under conditions sufficient to cleave the first product and the unreacted first substrate into a first set of fragments; and analyzing the first set of fragments by mass spectrometry (MS) to determine the quantity of the first product that was produced, wherein the quantity of the first product provides a measurement of the activity of the first enzyme in the sample. In some embodiments, the activity of the enzyme that is measured is a quantitative measurement of the activity, while in other embodiments, the measurement is qualitative. In various embodiments, the sample can contain a plurality of enzymes. Although many embodiments of the enzyme are described in the context of kinases, the invention can be used to assay other classes of enzymes, too.

In various cases, an internal standard of known mass can be added to the fragments prior to analysis by MS. In some cases, the internal standard is added prior to cleavage of the product and unreacted substrate into fragments. In some specific cases, the internal standard is an isotopically labeled substrate and/or product.

In certain cases, the sample is cell lysate from a human or animal subject and the human or animal subject is suspected of having a disease characterized by changes in the activity of an enzyme involved in a cellular process. In one embodiment, the disease suspected is cancer.

In some variations, the enzyme composition is a mixture of purified enzymes. The enzyme composition can also be all or a fraction of a cell lysate which contains enzymes from the cell. In certain cases, the lysate comes from a human or animal subject. The lysate may be of fewer than 100 cells, or fewer than 25 cells, or even fewer than 10 cells. In certain cases, the first enzyme is a kinase and, in specific embodiments, is a protein kinase. In some cases, the first enzyme is a protein modifying enzyme such as, but not limited to, an oxidoreductase, transferase, hydrolase, lyase, isomerase, or ligase.

In some cases, the methods disclosed herein may be used to measure the enzymatic activity of second enzyme. In some embodiments, the activity of the second enzyme is measured by using a first particle which has both a first substrate for the first enzyme and a second substrate for the second enzyme attached to it surface. In other embodiments, the activity of the second enzyme is measured by using a second particle having a second substrate attached to its surface. In yet other embodiments, the activity of the second enzyme can be measured by using a substrate that has a first domain that is a substrate domain for the first enzyme and a second domain that is a substrate domain for the second enzyme. In various embodiments, the first and second enzymatic reactions can occur under the same or different reaction conditions, and can be performed sequentially or simultaneously. The second product and unreacted second substrate can be cleaved and analyzed in a comparable manner as that of the first product and substrate. In some specific embodiments, the first and second enzyme are the same and that enzyme enzymatically modifies both a first substrate and a second substrate, which can be on the same or different particles, under the same or different incubating conditions. In the same fashion, the method can be performed to assay a third enzyme, a fourth enzyme, a fifth enzyme, and so on.

The enzyme being assessed in the disclosed methods can be any enzyme that modifies a substrate. In some variations, all of the enzymes to be assayed fall within the same class (e.g., protein kinases), whereas in other variations, enzymes of different classes are assayed together.

Another aspect of the invention is a method for screening compounds in order to identify a drug candidate comprising: measuring the activity of at least one enzyme from a biological sample, using a method described herein; and comparing the activity of the at least one enzyme in the presence and absence of the at least one test compound, wherein the method identifies an inhibitor or agonist drug candidate from reduced or increased activity, respectively, of the at least one enzyme in the presence of the at least one test compound. In certain cases, the method comprises measuring the activity of two or more enzymes in the presence or absence of a test compound. In various embodiments, the two or more enzymes are in the same signaling pathway, such as, for example, a pathway involved in cell growth, replication, differentiation, survival, or proliferation. Identification of a test compound as an inhibitor or an agonist of a particular enzyme or group of enzymes (as in the case of two or more enzymes being studied) can be accomplished by measuring the activity of a first enzyme or signaling pathway in the absence and presence of the test compound and comparing the activities as measured in order to assess the effect the test compound has. In certain cases, the methods can be used to assess the biological activity of the compound on non-target enzymes or pathways that may be relevant to drug metabolism/clearance, drug toxicity, and side-effects. This assessment may be useful for evaluating a compound as a potential drug candidate and/or its suitability for or efficacy in clinical trials. In some cases, the method comprises additional steps to further evaluate the compound. For example, the test compound is mixed with a pharmaceutically acceptable carrier to form a composition and the composition is administered to a subject to determine the effect of the composition in vivo. The subject can be a healthy subject for safety testing and/or a diseased subject and/or a model for a disease, for purpose of therapy or proving therapeutic efficacy. In one specific embodiment, the subject is a mammalian subject.

Another aspect of the invention is a method for screening an organism for a disease, disorder, or abnormality characterized by aberrant enzymatic activity comprising: quantitatively measuring the activity of an enzyme from a biological sample from an organism (e.g., a cell lysate from at least one cell of the organism) as described herein, and comparing the measurement to a reference measurement of the activity of the enzyme, wherein the presence or absence of the abnormality is identified from the comparison. Numerous enzyme-disease associations have been described in the literature and some are summarized below. Enzymes involved in cell growth, replication, differentiation, survival, or proliferation are only the preferred enzymes for such screening. In some cases, the cell lysate is obtained from a medical biopsy from a human and snap frozen to preserve enzymatic activity. In certain cases, the reference measurement is obtained from the same organism at a different time or from a different location in the organism. In other cases, the reference measurement is obtained from cells of the same cell type, from a different organism of the same species. In still other cases, the reference measurement is a statistical measurement calculated from measurements of samples of cells of the same cell type, from multiple organisms of the same species.

One continuing need in medicine, especially oncology and infectious diseases, is to be able to better characterize a disease in an individual patient to permit better selection of a medicament that is more likely to be therapeutically effective and/or have fewer side effects. Therefore, another aspect of the invention is a method of characterizing a disease, disorder, or abnormality comprising: quantitatively measuring the activity of at least one enzyme from a sample using any of the methods disclosed herein, wherein the sample comprises at least one cell known or suspected of being diseased isolated from a mammalian subject, or comprises a lysate of the at least one cell; comparing the measurement(s) to a reference measurement of the activity of the at least one enzyme; and characterizing the disease or disorder by identifying an enzyme with elevated activity in the at least one diseased cell compared to activity of the enzyme in non-diseased cells of the same type as the diseased cell. In certain cases, the disease is a neoplastic disease. In some embodiments, the method further comprises selecting a composition or compound for administration to the mammalian subject, wherein the composition or compound inhibits the activity of the enzyme with the elevated activity in the at least one diseased or neoplastic cell. In some cases, the method further comprises administering a composition or compound that inhibits the activity of the enzyme with the elevated activity in the at least one diseased or neoplastic cell. In certain cases, the method further comprises prescribing a medicament to the mammalian subject, wherein the medicament inhibits the activity of the enzyme with the elevated activity in the at least one diseased or neoplastic cell. In one specific embodiment, the mammalian subject is a human.

In some variations of the invention, the method is a method for screening for or diagnosing a disease state and the method includes a step of measuring enzyme activity as described herein in a biological sample from an organism, and a step of diagnosing the absence or the presence of the disease, or predisposition for the disease, by the measurement of enzyme activity. For example, a comparison of the measurement for a particular subject to measurements from other healthy subjects, or diseased subjects, of the same subject at an earlier point in time, indicates the proper conclusion about the disease state in the subject.

In some cases, the enzyme participates in a cellular signaling pathway. Cellular signaling pathways are the biochemical mechanisms by which cells convert extracellular signals into the required cellular response. Cellular signaling pathways are generally discussed in Hunter, “Signaling—2000 and Beyond,” Cell 100:113-117 (2000), the entirety of which is incorporated by reference herein. These signaling pathways involve a multitude of different enzymes and the methods disclosed herein can provide a measurement of the signaling pathway as a whole, not just of specific enzymes within the pathway. Some examples of signaling pathways, the activity of which can be measured using the methods disclosed herein, include PI3K/AKT pathways; Ras/Raf/MEK/Erk pathways; MAP kinase pathways; JAK/STAT pathways; mTOR/TSC pathways; heterotrimeric G protein pathways; PKA pathways; PLC/PKC pathways; NK-kappaB pathways; cell cycle pathways (cell cycle kinases); TGF-beta pathways; TLR pathways; Notch pathways; Wnt pathways; Nutrient signaling pathways (AMPK signaling); cell-cell and cell:substratum adhesion pathways (such as cadherin or integrins); stress signaling pathways (e.g., high/low salt, heat, radiation); cytokine signaling pathways; antigen receptor signaling pathways; and co-stimulatory immune signaling pathways. In some cases when the enzyme is involved in a cellular signaling pathway, the enzyme is an intracellular enzyme, i.e., an enzyme found only within a cell.

Additional features and variations of the invention will be apparent to those skilled in the art from the entirety of this application, including the drawing and detailed description, and all such features are intended as aspects of the invention. Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention.

In addition to the foregoing, the invention includes, as an additional aspect, all embodiments of the invention narrower in scope in any way than the variations specifically mentioned above. For example, although aspects of the invention may have been described by reference to a genus or a range of values for brevity, it should be understood that each member of the genus and each value or sub-range within the range is intended as an aspect of the invention. Likewise, various aspects and features of the invention can be combined, creating additional aspects which are intended to be within the scope of the invention. Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE DRAWINGS

FIG. 1 shows a schematic of one embodiments of the disclosed methods. A mixture of different protein substrates are immobilized to particles, which can be magnetic, which serve as substrates for kinases present in cell lysates. After in vitro kinase reactions, these protein substrates comprise phosphorylated sites that are the product of the kinase reactions. The particles are subsequently washed to remove components in cell lysates incompatible with subsequent steps. Protein substrates are then digested with a suitable protease, and the resultant phosphopeptides are quantified by LC-MS or related mass spectrometric method relative to their non-phosphorylated counterparts (for relative quantification) or to added internal standards incorporating isotope labels (for absolute quantification). In the scheme, IS refers to an internal standard.

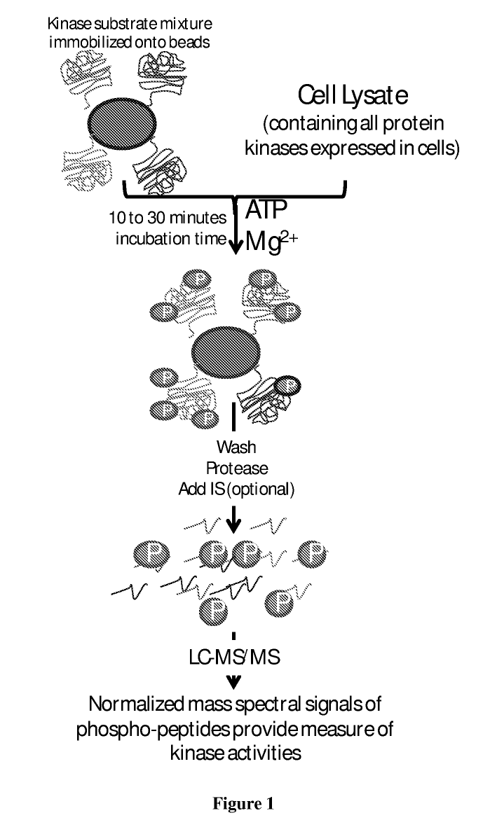

FIG. 2 shows data relating to multiplex quantification of kinase activities toward 4EBP1. The protein 4EBP1 was immobilized to beads and used for in vitro kinase reactions. NIH-3T3 cells were either left untreated or treated with 50 nM rapamycin (RAP), after which time they were lysed. Lysates were mixed with immobilized 4EBP1 and ATP/Mg²⁺. Reactions were performed at 37° C. for the indicated times and stopped by the addition of 0.1% formic acid. Particles were washed 3 times with 50 mM ammonium bicarbonate. Asp-N was added and the proteolytic reaction was allowed to occur overnight. Resultant peptides were analysed by LC-MS/MS for detection of phosphorylated peptides and by LC-MS for quantitation. The signals of the phosphopeptides shown (site of modification marked in bold underline) were normalized to the signals of their non-phosphorylated counterparts. The signal of the peptide containing pS111 was partially sensitive to RAP, whereas that of the peptide containing pS64 was totally abrogated by treatment of cells with the drug.

FIG. 3 shows data relating to multiplex quantification of kinase activities toward BAD. Starved NIH-3T3 cells, pre-treated or not with the inhibitors Wortmannin (WM) or PD98059 (PD), were stimulated with 10% fetal calf serum for 5 minutes prior to cell lysis. Cell lysates were mixed with immobilized BAD and ATP/Mg²⁺ and reactions allowed to occur at 37° C. for the indicated times. Reactions were stopped and beads washed to remove lysate components and to buffer exchange to 50 mM ammonium bicarbonate. Immobilized phosphorylated BAD was digested with trypsin and resultant peptides analysed by LC-MS and LC-MS/MS. The indicated phosphorylated peptides containing sites of phosphorylation pS170 and pS136 of the mouse BAD sequence showed increased signals as a function of reaction time. These observed activities increased when serum was added to the medium prior to lysis (compare black and grey data points) and were sensitive to the addition of WM but not PD (crosses and triangle data points, respectively), thus arguing that these activities are a measure of WM target inhibition, probably PI3K signalling activity, a known target of WM.

FIG. 4 shows (A) measured phosphorylation of an Aktide peptide (SEQ ID NO: 24) in the presence of cell lysate from Fuji cells using protocols described in WO 07/127,767 in the presence and absence of the kinase inhibitor LY294002; and (B) measured phosphorylation of immobilized substrates 4EBP1 and p53 in the presence and absence of the kinase inhibitor LY294002, using protocols of the invention described herein.

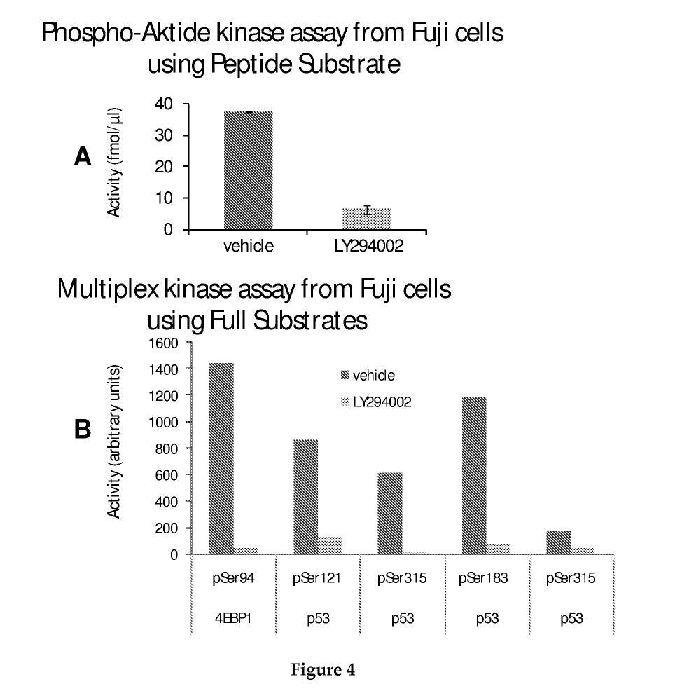

### DETAILED DESCRIPTION

Disclosed herein are methods of determining enzymatic activity using mass spectrometry. More particularly, methods are disclosed for determining enzymatic activity using enzyme substrates that are immobilized on a particle surface. The immobilized substrates are then contacted with an appropriate enzyme to produce immobilized products. The immobilized products are cleaved into fragments and can then be analyzed using mass spectrometry (MS). Prior methods used small peptide substrates in assays, but the disclosed method can easily employ substrates of any size. For example, the method uses larger substrates (e.g., full protein substrates, or full domain substrates), because the substrate, and resulting products, are immobilized on a particle. Due to this immobilization, they can be isolated from the sample and from any biological material in the reaction, then cleaved into suitable length fragments for MS analysis.

It was reasoned that immobilized substrates for enzymes that closely mimic wild-type substrates, such as full-length proteins, domains having a reactive site for an enzyme, and the like, can serve as ideal substrates for mass spectrometry-based analysis of enzyme activities, compared to short peptides that merely included a small portion of the reactive site. This is because the physiological substrates for enzymes, e.g., protein kinases, are typically full-length proteins rather than small peptides. Thus, the disclosed approach provides for a more efficient and specific way of measuring enzyme, e.g., kinase, activities. Moreover, proteins contain several sites of activity, such as phosphorylation sites, and therefore using larger substrates allows for measuring several enzymes' kinase activities simultaneously. This feature is important as it allows for a more comprehensive view of signaling pathway activation, and to assess in an unbiased way the interplay of signaling pathways within the network.

The immobilization of a substrate to a particle allows for practical manipulation of enzyme substrates, and facilitates the removal of impurities, extraneous proteins, detergents and other reagents needed for enzymatic reactions but not compatible with mass spectrometry analysis, making the approach amenable to automation and facilitating its implementation and throughput. Table 1 shows a comparison of the different mass spectrometry-based methods that have been developed to date to quantify cell signaling.

TABLE 1

| | Method name | | | | | | |
|---|---|---|---|---|---|---|---|
| | | | ICAT/ | | Chemical | Small Peptide | Immobilized |
| Property of the method | SILAC | PAIS | iTRAQ | AQUA | tagging | Substrate | Substrate |
| Absolute quantification | NO | NO | NO | YES | NO | YES | YES |
| of cell signalling activity | | | | | | | |
| Multiplex quantification | YES | YES | NO | YES | NO | YES | YES |
| of cell signalling activity | | | | | | | |
| Amplification of signal | NO | NO | NO | NO | NO | YES | YES |
| Number of cells needed | ~10⁷ | ~10⁷ | ~10⁷ | ~10⁷ | ~10⁷ | 10 to 1000 | 10 to 1000 |
| for analysis | | | | | | | |
| Suitable for primary | NO | NO | NO | NO* | NO | YES | YES |
| tissues & biopsies | | | | | | | |
| Suitable for the discovery | YES | YES | YES | NO | YES | YES | YES |
| of new activities | | | | | | | |
| Reference | Olsen et al., | Cutillas et al, | Gygi et al., | Gerber, et al., | Barglow et al., | Cutillas et al., | Disclosed |
| | Cell, | Mol. Cell | Nat Biotechnol, | Proc Natl Acad | Nat Methods, | Proc Natl Acad | herein |
| | 127: 635-48 | Proteomics, | 17: 994-9 (1999) | Sci USA, | 4: 822-7 | Sci USA | |
| | (2006) | 4: 1038-51 | and Ross et al., | 100: 6940-5 | (2007) | 103: 8959-64 | |
| | | (2005) | Mol Cell | (2003) | | (2006) and WO | |
| | | | Proteomics, | | | 07/127767 | |
| | | | 3: 1154-69 (2004) | | | | |

*AQUA may be suitable for the analysis of primary tissues when the target protein is high abundant, but it is not suitable for the quantification of phosphorylated peptides because these are normally present in low copy numbers

The term “enzyme” refers to any protein that has a biological activity of modifying, or catalyzing the modification of, a molecule (referred to herein as a “substrate”) into another molecule or molecules (referred to herein as a “product”). Typically, the product and substrate will have a different molecular weight. For example, a kinase is an enzyme that modifies a substrate molecule by adding a phosphate moiety, to create a phosphorylated product molecule. Kinases can be protein kinases, lipid kinases, carbohydrate kinases such as phosphofructokinase, or small molecule kinases such as pyruvate kinase. Exemplary protein kinases which may be used in the disclosed methods are listed below in Table 2. An enzyme may include one or more polypeptide chains as well as modifications (e.g., glycosylation, phosphorylation, methylation, etc.) or co-factors (e.g., metal ions).

Unless context clearly dictate to the contrary, the articles “all” or “an” should be construed (especially in the claims) to refer to one or more. For example, the term “an enzyme” in the preceding description of the method refers to one or more enzymes. As described in greater detail below, the method can be practiced in a multiplex fashion to analyze the activity of multiple enzymes at once. Each enzyme modifies (e.g., catalyzes the modification of) a substrate to form a product. The use of ordinals (e.g., “first” or “second” or “third” and so forth) to refer to elements such as an enzyme, a substrate, a standard, or a product is for clarity purposes only, to identify which enzyme, substrate, product, and standard are related to each other and to distinguish the substrate, standard, and product of one enzyme from the substrate, product, and standard of another enzyme that is assayed. The ordinals are not meant to imply any particular relationship or required order between the multiple enzymes that are to be assayed.

Enzymes that may be evaluated using the techniques and methods disclosed herein include any enzyme involved in a cellular process, more specifically, enzymes such as kinases, phosphatases, oxidoreductases, transferases, hydrolases, lyases, isomerases, and ligases. In some preferred embodiments, kinases are assayed. More specifically, both protein kinases and lipid kinases may be evaluated. Other enzymes such as lipid kinases (e.g., phosphoinositide 3-kinase) can also be assayed when they have protein kinase activity.

Specific kinases contemplated for assay according to the methods disclosed herein include those listed in Table 2. Nonlimiting examples of contemplated kinase families include phosphoinositide kinases, the cyclic nucleotide regulated protein kinase family, the diacylglycerol-activated, phospholipid-dependent protein kinase C (PKC) family, the RAC (Akt) protein kinase family, the family of kinases that phosphorylate G protein-coupled receptors, the budding yeast AGC-related protein kinase family, the kinases that phosphorylate ribosomal protein S6 family, the budding yeast DBF2/20 family, the flowering plant PVPK1 protein kinase homolog family, the kinases regulated by Ca2+/CaM and close relatives family, the KIN1/SNF1/Nim1 family, the cyclin-dependent kinases (CDKs) and close relatives family, the ERK (MAP) kinase family, the glycogen synthase kinase 3 (GSK3) family, the casein kinase II family, the Clk family, the Src family, the Tec/Atk family, the Csk family, the Fes (Fps) family, the Syk/ZAP70 family, the Tyk2/Jak1 family, the Ack family, the Focal adhesion kinase family, the Epidermal growth factor receptor family, the Eph/Elk/Eck orphan receptor family, the Axl family, the Tie/Tck family, the Platelet-derived growth factor receptor family, the Fibroblast growth factor receptor family, the Insulin receptor family, the LTK/ALK family, the Ros/Sevenless family, the Trk/Ror family, the DDR/TKT family, the Hepatocyte growth factor family, the Nematode Kin15/16 family, the Polo family, the MEK/STE7 family, the PAK/STE20 family, the MEKK/STE11 family, the NimA family, the wee1/mik1 family, Kinases involved in transcriptional control family, the Activin/TGFb receptor family, the Flowering plant putative receptor kinases and close relatives family, the PSK/PTK “mixed lineage” leucine zipper domain family, the Casein kinase I family, and the PKN prokaryotic protein kinase family.

Resources for information about kinases include Genbank, the Swiss-Protein protein knowledge database, the protein kinase resource database on the worldwide web at http://www.kinasenet.org/pkr/Welcome.do, the worldwide web database at www.kinase.com, and numerous other paper and electronic resources.

Individual kinases contemplated for analysis in the disclosed methods include, but are not limited to, PIK3CA, PIK3CB, PIK3CG, PIK3CD, cAPKα, cAPKβ, cAPKγ, EcAPKα, DC0, DC1, DC2, ApIC, SAK, DdPK1, DdPk2, TPK1, TPK2, TPK3, PKG-I, PKG-II, DG1, DG2, PKCα, PKCβ, PKCγ, DPKC53b, DPKC53e, ApII, PKCd, PKCe, PKCet, PKCth, DPKC98, ApIII, CeTPA1, CePKC1B, PKC1, pck1+, pck2+, PKCz, PKCi, PKCm, Akt1, Akt2, SmRAC, bARK1, bARK2, RhoK, GRK5, IT11, GRK6, DmGPRK1, FmGPRK2, SCH9, YPK1, YKR2, S6K, RSK1N, RSK2N, DBF2, DBF20, PVPK1, G11A, ZmPPK, ATPK5, ATPK7, ATPK64, PsPK5, DM, Sgk, Mast205, SPK1, CaMKIIα, CaMKIIβ, CaMKIIγ, CaMKIIδ, DmCamKII, CamKI, CaMKIV, DdMKCK, DUN1, PSK-H1, CMK1, CMK2, ACMPK, MLCK-K, MLCK-M, Titwn, TWITCH, MRE4, PhKgM, PhKgT, RSK1C, RSK2C, ASK1, ASK2, CDPK, AK1, OsSPK, KIN1, KIN2, kin1+, p78, SNF1, RKIN1, AKIN10, BKIN12, WPK4, nim1+, YKL453, YCL24, MAPKAP2, PfCPK, PfPK2, CDC2Hs, Cdk2, Cdk3, Cdk4, Cdk5, Cdk6, PCTAIRE1, PCTAIRE2, PCTAIRE3, CAK/MO15, Dm2, Dm2C, Ddcdc2, DdPRK, LmmCRK1, PfC2R, EhC2R, CfCdc2R, cdc2+, CDC28, PHO85, KIN28, FpCdc2, MsCdc2b, OsC2R, ERK1, ERK2, ERK3, Jnk1, FmERKA, CeMPK1, CaERK1, KSS1, FUS3, HOG1, SLT2, spk1+, FpERK1, NTF3, FpMPK1, FpMPK2, FpMPK3, FpMPK4, FpMPK5, FpMPK6, FpMPK7, GSK3a, GSK2b, Sgg/zw3, MCK, MDS1, ASK-a, ASK-g, CKIIa, CKIIa′, DmCKII, CeCKII, TpCKII, DdCKIIa, CKA1, CKA2, SpCka1, GpCKII, CIk, PSK-G1, Doa, KNS1, PSK-H2, YAK1, dsk1+, prp1+, GTAp58, Dcdrk, CHED, CTK1, SGV1, KKIALRE, MAK, SME1, csk1+, MHK, c-Src, c-Yes, FYN, YRK, c-Fgr, LYN, HCK, LCK, BLK, TorFYK, Dsrc64, STK, SRK1, SRK2, SRK3, SRK4, Tex, Itk/Tsk, Btk, Dsrc28, DtSpk-1, Csk, Matk, c-Fes, FER, Dfps, PTK Group V, Abl, c-Abl, ARG, Dab1, Nab1, Syk2, ZAP70, Htk16, TYK2, JAK1, JAK2, HOP, ACK, GAK, EGFR, ErbB2, ErbB3, ErbB4, DER, let-23, SER, ECK, EEK, HEK, Ehk-1, Ehk-2, SEK, ELK, Cek10, Cek9, HEK2, Buk, EPH, Azl, Ark, c-Eyk, Brt/Sky, TiE, Tek, PDGFR-α, PDGFR-β, CSF1R, c-kit, Flk2, Flt1, Flt4, Flk1, Fig, Bek, FGFR-3, FGFR-4, DFGFR, INS.r, IRR, IGF1R, DILR, LTK, ALK, c-ros, 7LESS, Trk, TrkB, TrkC, TorRTK, Ror1, Ror2, Dror, DDR, TKT, MET, c-Sea, RON, Nkin15, Nkin16, RET, KLG, Nyk/RYK, TORSO, Dtrk, Plk, SNK, polo, CDC5, MEK1, MEK2, Dsor1, PBS2, wis1+, MKK1, MKK2, byr1+, STE7, PAK, STE20, MEKK, STE11, byr2, BCK1, NPK1, Mek1, MrkA, nimA, KIN3, FUSED, wee1+, mik1+. HsWee1, HRI, PKR, GCN2, c-raf, Araf, Braf, DmRaF, CeRaf, Ctrl, TGFbRII, ActRIIA, ActRIIB, TSR-1, TskL7, ALK-3, ALK-4, ALK-5, ALK-6, C14, Daft, Daf4, DmAtr-II, DmSax, SR2, SR6, Pto, TMK1, APK1, NAK, ZMPK1, PRO25, TMK1, pelle, MLK1, PTK1, CKIa, CKIb, CKId, TCK1, YCK2, HRR25, PKN1, PKN2, IRE1, CDC7, COT, YpkA, ninaC, CDC15, chk1+, NPR1, TSL, PIM1, ran1+, TTK, ELM1, VPS15, YKL516, c-mos, Pstk1, DPYK1, DPYK2, PhyCer, and GmPK6.

TABLE 2
SwissProt Accession Numbers and abbreviated gene names of exemplary protein kinases

| | | |
|---|---|---|
| P36896, ACV1B_HUMAN | Q52EB3, ATG1_MAGGR | Q9Z1S0, BUB1B_MOUSE |
| Q61271, ACV1B_MOUSE | Q7RX99, ATG1_NEUCR | O43683, BUB1_HUMAN |
| P80202, ACV1B_RAT | Q8TFN2, ATG1_PICAN | O08901, BUB1_MOUSE |
| P37023, ACVL1_HUMAN | Q8TGI1, ATG1_PICPA | O94751, BUB1_SCHPO |
| Q61288, ACVL1_MOUSE | Q9Y7T4, ATG1_SCHPO | P41695, BUB1_YEAST |
| P80203, ACVL1_RAT | Q6C7U0, ATG1_YARLI | Q9GKI7, C43BP_BOVIN |
| Q28041, ACVR1_BOVIN | P53104, ATG1_YEAST | Q9Y5P4, C43BP_HUMAN |
| Q04771, ACVR1_HUMAN | Q9M3G7, ATM_ARATH | Q9EQG9, C43BP_MOUSE |
| P37172, ACVR1_MOUSE | Q13315, ATM_HUMAN | P43568, CAK1_YEAST |
| P80201, ACVR1_RAT | Q62388, ATM_MOUSE | Q754N7, CBK1_ASHGO |
| Q28043, ACVR2_BOVIN | Q6PQD5, ATM_PIG | Q6FP74, CBK1_CANGA |
| P27037, ACVR2_HUMAN | Q13535, ATR_HUMAN | Q6BLJ9, CBK1_DEBHA |
| P27038, ACVR2_MOUSE | Q9JKK8, ATR_MOUSE | P31034, CBK1_KLULA |
| P38444, ACVR2_RAT | Q96GD4, AURKB_HUMA | Q6TGC6, CBK1_PNECA |
| Q28560, ACVR2_SHEEP | O70126, AURKB_MOUSE | Q6CFS5, CBK1_YARLI |
| P27039, ACVR2_XENLA | Q9N0X0, AURKB_PIG | P53894, CBK1_YEAST |
| P54741, AFSK_STRCO | O55099, AURKB_RAT | P38973, CC2H1_TRYBB |
| P54742, AFSK_STRGR | Q9UQB9, AURKC_HUMA | P54664, CC2H1_TRYCO |
| P38080, AKL1_YEAST | O88445, AURKC_MOUSE | P54665, CC2H2_TRYBB |
| Q01314, AKT1_BOVIN | Q95126, AVR2B_BOVIN | P54666, CC2H3_TRYBB |
| P31749, AKT1_HUMAN | Q13705, AVR2B_HUMAN | P21127, CD2L1_HUMAN |
| P31750, AKT1_MOUSE | P27040, AVR2B_MOUSE | P24788, CD2L1_MOUSE |
| P47196, AKT1_RAT | P38445, AVR2B_RAT | P46892, CD2L1_RAT |
| P31751, AKT2_HUMAN | P27041, AVR2B_XENLA | Q9UQ88, CD2L2_HUMAN |
| Q60823, AKT2_MOUSE | Q94F62, BAK1_ARATH | Q14004, CD2L5_HUMAN |
| P47197, AKT2_RAT | Q01389, BCK1_YEAST | Q69ZA1, CD2L5_MOUSE |
| Q9Y243, AKT3_HUMAN | Q9NSY1, BMP2K_HUMA | Q9BWU1, CD2L6_HUMA |
| Q9WUA6, AKT3_MOUSE | Q91Z96, BMP2K_MOUSE | Q9NYV4, CD2L7_HUMAN |
| Q63484, AKT3_RAT | Q13873, BMPR2_HUMAN | P24923, CDC21_MEDSA |
| Q96Q40, AL2S7_HUMAN | O35607, BMPR2_MOUSE | P29618, CDC21_ORYSA |
| Q16671, AMHR2_HUMAN | P36894, BMR1A_HUMAN | P19026, CDC21_PEA |
| Q62893, AMHR2_RAT | P36895, BMR1A_MOUSE | P35567, CDC21_XENLA |
| P10398, ARAF_HUMAN | Q05438, BMR1B_CHICK | Q05006, CDC22_MEDSA |
| P04627, ARAF_MOUSE | O00238, BMR1B_HUMAN | P29619, CDC22_ORYSA |
| O19004, ARAF_PIG | P36898, BMR1B_MOUSE | P28567, CDC22_PEA |
| P14056, ARAF_RAT | Q04982, BRAF1_CHICK | P24033, CDC22_XENLA |
| O59790, ARK1_SCHPO | P34908, BRAF1_COTJA | P43063, CDC28_CANAL |
| P43291, ASK1_ARATH | P15056, BRAF1_HUMAN | P00546, CDC28_YEAST |
| P43292, ASK2_ARATH | P28028, BRAF1_MOUSE | Q38772, CDC2A_ANTMA |
| Q75CH3, ATG1_ASHGO | O22476, BRI1_ARATH | P24100, CDC2A_ARATH |
| Q6H9I1, ATG1_BOTCI | Q8GUQ5, BRI1_LYCES | Q38773, CDC2B_ANTMA |
| Q5A649, ATG1_CANAL | Q8L899, BRI1_LYCPE | P25859, CDC2B_ARATH |
| Q6FL58, ATG1_CANGA | Q9ZWC8, BRL1_ARATH | Q38774, CDC2C_ANTMA |
| P87248, ATG1_COLLN | Q9ZPS9, BRL2_ARATH | P23573, CDC2C_DROME |
| Q5K8D3, ATG1_CRYNE | Q9LJF3, BRL3_ARATH | Q38775, CDC2D_ANTMA |
| Q6BS08, ATG1_DEBHA | Q8TDC3, BRSK1_HUMAN | Q01917, CDC2H_CRIFA |
| Q5BCU8, ATG1_EMENI | Q8IWQ3, BRSK2_HUMAN | P34117, CDC2H_DICDI |
| Q6CSX2, ATG1_KLULA | O60566, BUB1B_HUMAN | P61075, CDC2H_PLAF7 |
| Q07785, CDC2H_PLAFK | P51953, CDK7_CARAU | O22932, CPK11_ARATH |
| P54119, CDC2_AJECA | P54685, CDK7_DICDI | P92937, CPK15_ARATH |
| P48734, CDC2_BOVIN | P50613, CDK7_HUMAN | Q8NK05, CPK1_CRYNE |
| P34556, CDC2_CAEEL | Q03147, CDK7_MOUSE | Q9LDI3, CPK24_ARATH |
| P51958, CDC2_CARAU | P51952, CDK7_RAT | Q06309, CRK1_LEIME |
| P93101, CDC2_CHERU | P20911, CDK7_XENLA | Q12126, CRK1_SCHPO |
| P13863, CDC2_CHICK | Q9VT57, CDK8_DROME | P36615, CSK1_SCHPO |
| P34112, CDC2_DICDI | P49336, CDK8_HUMAN | Q08467, CSK21_ARATH |
| P23572, CDC2_DROME | P46551, CDK9_CAEEL | P68399, CSK21_BOVIN |
| Q00646, CDC2_EMENI | P50750, CDK9_HUMAN | P21868, CSK21_CHICK |
| P06493, CDC2_HUMAN | Q99J95, CDK9_MOUSE | P68400, CSK21_HUMAN |
| P23111, CDC2_MAIZE | Q641Z4, CDK9_RAT | Q60737, CSK21_ MOUSE |
| P11440, CDC2_MOUSE | Q96WV9, CDK9_SCHPO | P33674, CSK21_RABIT |
| Q9DGA5, CDC2_ORYCU | O76039, CDKL5_HUMAN | P19139, CSK21_RAT |
| Q9DGA2, CDC2_ORYJA | P62344, CDPK1_PLAF7 | P15790, CSK21_YEAST |
| Q9DGD3, CDC2_ORYLA | P62343, CDPK1_PLAFK | Q08466, CSK22_ARATH |
| Q9DG98, CDC2_ORYLU | Q7RAH3, CDPK1_PLAYO | P20427, CSK22_BOVIN |
| P43290, CDC2_PETHY | Q8ICR0, CDPK2_PLAF7 | P21869, CSK22_CHICK |
| Q9W739, CDC2_RANDY | O15865, CDPK2_PLAFK | P19784, CSK22_HUMAN |
| P39951, CDC2_RAT | Q9NJU9, CDPK3_PLAF7 | O54833, CSK22_MOUSE |
| P04551, CDC2_SCHPO | Q7RAV5, CDPK3_PLAYO | P28020, CSK22_XENLA |
| Q41639, CDC2_VIGAC | P62345, CDPK4_PLABA | P19454, CSK22_YEAST |
| P52389, CDC2_VIGUN | Q8IBS5, CDPK4_PLAF7 | O64817, CSK23_ARATH |
| P32562, CDC5_YEAST | Q7RJG2, CDPK4_PLAYO | P18334, CSK2A_CAEEL |
| P06243, CDC7_YEAST | Q09170, CDS1_SCHPO | Q02720, CSK2A_DICDI |
| Q15131, CDK10_HUMAN | P38938, CEK1_SCHPO | P08181, CSK2A_DROME |
| P43450, CDK2_CARAU | O14757, CHK1_HUMAN | P28523, CSK2A_MAIZE |
| O55076, CDK2_CRIGR | O35280, CHK1_MOUSE | Q8TG13, CSK2A_NEUCR |
| Q04770, CDK2_ENTHI | P34208, CHK1_SCHPO | P40231, CSK2A_SCHPO |
| P24941, CDK2_HUMAN | P38147, CHK1_YEAST | O76484, CSK2A_SPOFR |
| P48963, CDK2_MESAU | Q9U1Y5, CHK2_CAEEL | P28547, CSK2A_THEPA |
| P97377, CDK2_MOUSE | O96017, CHK2_HUMAN | Q05609, CTR1_ARATH |
| Q63699, CDK2_RAT | Q9Z265, CHK2_MOUSE | O14578, CTRO_HUMAN |
| P23437, CDK2_XENLA | Q8RWC9, C1PK1_ARATH | P49025, CTRO_MOUSE |
| Q00526, CDK3_HUMAN | Q6X4A2, CIPK1_ORYSA | P27450, CX32_ARATH |
| P11802, CDK4_HUMAN | Q9HFW2, CLA4_ASHGO | P20792, DAF1_CAEEL |
| P30285, CDK4_MOUSE | O14427, CLA4_CANAL | P50488, DAF4_CAEEL |
| P79432, CDK4_PIG | P48562, CLA4_YEAST | P53355, DAPK1_HUMAN |
| P35426, CDK4_RAT | P49759, CLK1_HUMAN | Q80YE7, DAPK1_MOUSE |
| Q91727, CDK4_XENLA | P22518, CLK1_MOUSE | Q9UIK4, DAPK2_HUMAN |
| Q02399, CDK5_BOVIN | P49760, CLK2_HUMAN | Q8VDF3, DAPK2_MOUSE |
| P48609, CDK5_DROME | O35491, CLK2_MOUSE | O43293, DAPK3_HUMAN |
| Q00535, CDK5_HUMAN | P49761, CLK3_HUMAN | O54784, DAPK3_MOUSE |
| P49615, CDK5_MOUSE | O35492, CLK3_MOUSE | O88764, DAPK3_RAT |
| Q03114, CDK5_RAT | Q63117, CLK3_RAT | P32328, DBF20_YEAST |
| P51166, CDK5_XENLA | Q9HAZ1, CLK4_HUMAN | P22204, DBF2_YEAST |
| Q00534, CDK6_HUMAN | O35493, CLK4_MOUSE | O15075, DCAK1_HUMAN |
| Q64261, CDK6_MOUSE | P38679, COT1_NEUCR | Q9JLM8, DCAK1_MOUSE |
| O08875, DCAK1_RAT | Q9WUM7, HIPK2_MESAU | Q6L8L1, KAIC_ACAMR |
| Q8N568, DCAK2_HUMAN | Q9QZR5, HIPK2_MOUSE | Q8YT40, KAIC_ANASP |
| Q6PGN3, DCAK2_MOUSE | Q9H422, HIPK3_HUMAN | Q7VAN5, KAIC_PROMA |
| P49762, DOA_DROME | Q9ERH7, HIPK3_MOUSE | Q7V5W7, KAIC_PROMM |
| Q9Y2A5, DUET_HUMAN | O88850, HIPK3_RAT | Q7V0C4, KAIC_PROMP |
| P39009, DUN1_YEAST | Q8T0S6, HIPPO_DROME | Q79V60, KAIC_SYNEL |
| Q9Y463,DYR1B_HUMAN | Q750A9, HOG1_ASHGO | Q8GGL1, KAIC_SYNLI |
| Q9Z188, DYR1B_MOUSE | Q92207, HOG1_CANAL | Q6L8L5, KAIC_SYNP2 |
| Q9V3D5, DYRK2_DROME | Q6FIU2, HOG1_CANGA | Q79PF4, KAIC_SYNP7 |
| Q92630, DYRK2_HUMAN | Q9UV50, HOG1_DEBHA | Q8VL13, KAIC_SYNP8 |
| Q9BQI3, E2AK1_HUMAN | P32485, HOG1_YEAST | Q7U8R3, KAIC_SYNPX |
| Q9Z2R9, E2AK1_MOUSE | O93982, HOG1_ZYGRO | Q6L8J9, KAIC_SYNVU |
| P33279, E2AK1_RABIT | Q08732, HRK1_YEAST | P74646, KAIC_SYNY3 |
| Q63185, E2AK1_RAT | P50582, HSK1_SCHPO | Q10078, KAND_SCHPO |
| Q9P2K8, E2AK4_HUMAN | P57058, HUNK_HUMAN | P06244, KAPA_YEAST |
| Q9QZ05, E2AK4_MOUSE | O88866, HUNK_MOUSE | P05131, KAPB1_BOVIN |
| P32801, ELM1_YEAST | Q68UT7, HUNK_PANTR | P24256, KAPB2_BOVIN |
| P28869, ERK1_CANAL | Q9UPZ9, ICK_HUMAN | P40376, KAPB_SCHPO |
| P42525, ERK1_DICDI | Q9JKV2, ICK_MOUSE | P06245, KAPB_YEAST |
| P40417, ERKA_DROME | Q62726, ICK_RAT | P00517, KAPCA_BOVIN |
| O75460, ERN1_HUMAN | Q6CWQ4, ICL1_KLULA | Q8MJ44, KAPCA_CANFA |
| Q9EQY0, ERN1_MOUSE | Q9VEZ5, IKKB_DROME | P25321, KAPCA_CRIGR |
| Q76MJ5, ERN2_HUMAN | O14920, IKKB_HUMAN | P17612, KAPCA_HUMAN |
| Q9Z2E3, ERN2_MOUSE | O88351, IKKB_MOUSE | P05132, KAPCA_MOUSE |
| Q9LYN8, EXS_ARATH | Q9QY78, IKKB_RAT | P36887, KAPCA_PIG |
| Q9NLA1, FLR4_CAEEL | Q13418, ILK1_HUMAN | P27791, KAPCA_RAT |
| P16892, FUS3_YEAST | P57043, ILK2_HUMAN | Q9MZD9, KAPCA_SHEEP |
| P23647, FUSED_DROME | P57044, ILK_CAVPO | P68180, KAPCB_CRIGR |
| Q9P7J8, GAD8_SCHPO | O55222, ILK_MOUSE | P22694, KAPCB_HUMAN |
| Q9LX30, GCN2_ARATH | Q755C4, IPL1_ASHGO | P68181, KAPCB_MOUSE |
| Q9HGN1, GCN2_SCHPO | Q59S66, IPL1_CANAL | P05383, KAPCB_PIG |
| P15442, GCN2_YEAST | Q6FV07, IPL1_CANGA | P68182, KAPCB_RAT |
| Q12263, GIN4_YEAST | Q6BVA0, IPL1_DEBHA | P22612, KAPCG_HUMAN |
| O61661, GRP_DROME | Q6C3J2, IPL1_YARLI | O62846, KAPCG_MACMU |
| P49840, GSK3A_HUMAN | P38991, IPL1_YEAST | P49673, KAPC_ASCSU |
| P18265, GSK3A_RAT | P51617, IRAK1_HUMAN | P21137, KAPC_CAEEL |
| P49841, GSK3B_HUMAN | Q62406, IRAK1_MOUSE | P34099, KAPC_DICDI |
| Q9WV60, GSK3B_MOUSE | Q9NWZ3, IRAK4_HUMAN | P12370, KAPC_DROME |
| P18266, GSK3B_RAT | Q8R4K2, IRAK4_MOUSE | Q8SRK8, KAPC_ENCCU |
| P51136, GSK3H_DICDI | P32361, IRE1_YEAST | P05986, KAPC_YEAST |
| P83101, GSK3H_DROME | Q9U6D2, JNK1_ANCCA | P21901, KAPL_APLCA |
| P38970, HALS_YEAST | Q8WQG9, JNK1_CAEEL | P38070, KBN8_YEAST |
| P83103, HASP_DROME | P92208, JNK_DROME | Q9UU87, KC61_SCHPO |
| Q8TF76, HASP_HUMAN | Q966Y3, JNK_SUBDO | P25389, KCC4_YEAST |
| Q9Z0R0, HASP_MOUSE | Q09792, KAA8_SCHPO | Q10364, KDBE_SCHPO |
| Q86Z02, HIPK1_HUMAN | Q09815, KAB7_SCHPO | P16911, KDC1_DROME |
| O88904, HIPK1_MOUSE | P31374, KAB7_YEAST | O14019, KDPG_SCHPO |
| Q9H2X6, HIPK2_HUMAN | Q09831, KADS_SCHPO | P53233, KG1Z_YEAST |
| P00516, KGP1A_BOVIN | Q9VPC0, KPEL_DROME | P09296, KR2_VZVD |
| Q13976, KGP1A_HUMAN | Q19266, KPC3_CAEEL | P54644, KRAC_DICDI |
| O77676, KGP1A_RABIT | P83099, KPC4_DROME | Q07292, KRAF1_CAEEL |
| P21136, KGP1B_BOVIN | P04409, KPCA_BOVIN | P11346, KRAF1_DROME |
| P14619, KGP1B_HUMAN | P17252, KPCA_HUMAN | O57259, KRB2_VACCA |
| Q9Z0Z0, KGP1B_MOUSE | P20444, KPCA_MOUSE | P21098, KRB2_VACCC |
| Q03042, KGP1_DROME | P10102, KPCA_RABIT | P24362, KRB2_VACCV |
| Q03043, KGP24_DROME | P05696, KPCA_RAT | Q15418, KS6A1_HUMAN |
| P32023, KGP25_DROME | P05126, KPCB_BOVIN | P18653, KS6A1_MOUSE |
| Q13237, KGP2_HUMAN | P05771, KPCB_HUMAN | Q63531, KS6A1_RAT |
| Q61410, KGP2_MOUSE | P68404, KPCB_MOUSE | Q15349, KS6A2_HUMAN |
| Q64595, KGP2_RAT | P05772, KPCB_RABIT | Q9WUT3, KS6A2_MOUSE |
| P43637, KGS9_YEAST | P68403, KPCB_RAT | P51812, KS6A3_HUMAN |
| P38692, KIC1_YEAST; | P05128, KPCG_BOVIN | P18654, KS6A3_MOUSE |
| P40494, KIJ5_YEAST | P05129, KPCG_HUMAN | O75676, KS6A4_HUMAN |
| Q38997, KIN10_ARATH | P63318, KPCG_MOUSE | Q9Z2B9, KS6A4_MOUSE |
| P92958, KIN11_ARATH | P10829, KPCG_RABIT | O75582, KS6A5_HUMAN |
| P06242, K1N28_YEAST | P63319, KPCG_RAT | Q8C050, KS6A5_MOUSE |
| P13186, KIN2_YEAST | Q90XF2, KPCI_BRARE | Q9UK32, KS6A6_HUMAN |
| P22209, KIN3_YEAST | P41743, KPCI_HUMAN | P18652, KS6AA_CHICK |
| Q01919, KIN4_YEAST | Q62074, KPCI_MOUSE | P10665, KS6AA_XENLA |
| P25341, K1N82_YEAST | Q5R4K9, KPCI_PONPY | P10666, KS6AB_XENLA |
| P00513, KIPA_BPT7 | Q05513, KPCZ_HUMAN | Q21734, KS6A_CAEEL |
| O74526, KJ45_SCHPO | Q02956, KPCZ_MOUSE | P23443, KS6B1_HUMAN |
| P47042, KJF7_YEAST; | O19111, KPCZ_RABIT | Q8BSK8, KS6B1_MOUSE |
| Q9HFF4, KK31_SCHPO | P09217, KPCZ_RAT | P67998, KS6B1_RABIT |
| Q9P6P3, KKB3_SCHPO | Q05652, KPEL_DROME | P67999, KS6B1_RAT |
| Q8N5S9,KKCC1_HUMAN | Q39030, KPK19_ARATH | Q9UBS0, KS6B2_HUMAN |
| Q8VBY2, KKCC1_MOUSE | P42818, KPK1_ARATH | Q9Z1M4, KS6B2_MOUSE |
| P97756, KKCC1_RAT | Q02595, KPK2_PLAFK | Q12701, KSG1_SCHPO |
| Q96RR4, KKCC2_HUMAN | Q05999, KPK7_ARATH | P38691, KSP1_YEAST |
| Q8C078, KKCC2_MOUSE | P17801, KPRO_MAIZE | P14681, KSS1_YEAST |
| O88831, KKCC2_RAT | P11801, KPSH1_HUMAN | O95835, LATS1_HUMAN |
| Q9UTH3, KKE1_SCHPO | Q08097, KR1_BHV1S | Q8BYR2, LATS1_MOUSE |
| Q00532, KKIA_HUMAN | Q04543, KR1_CHV9D | Q9NRM7, LATS2_HUMA |
| P34244, KKK1_YEAST | P28926, KR1_EHV1B | Q7TSJ6, LATS2_MOUSE |
| P28708, KKL6_YEAST | P32516, KR1_EHV1K | P53667, LIMK1_HUMAN |
| P36005, KKQ1_YEAST | P84390, KR1_EHV1V | P53668, LIMK1_MOUSE |
| P36004, KKQ8_YEAST | P04413, KR1_HHV11 | P53669, LIMK1_RAT |
| P36003, KKR1_YEAST | P13287, KR1_HHV2H | Q10156, LKH1_SCHPO |
| Q03533, KM8S_YEAST | P17613, KR1_PRVKA | O61267, LOK_DROME |
| P53739, KN8R_YEAST | P24381, KR1_PRVN3 | Q02779, M3K10_HUMAN |
| P53974, KNC0_YEAST | P09251, KR1_VZVD | Q16584, M3K11_HUMAN |
| P32350, KNS1_YEAST | P13288, KR2_EBV | Q12852, M3K12_HUMAN |
| Q08217, KOES_YEAST | P28966, KR2_EHV1B | Q60700, M3K12_MOUSE |
| Q9Y7J6, KOIA_SCHPO | P84391, KR2_EHV1V | Q63796, M3K12_RAT |
| Q12236, KOK0_YEAST | P04290, KR2_HHV11 | O43283, M3K13_HUMAN |
| Q12222, KOM8_YEAST | P30662, KR2_PRVN3 | Q5R8X7, M3K13_PONPY |
| Q99558, M3K14_HUMAN | Q7KZI7,MARK2_HUMAN | P49186, MK09_RAT |
| Q9WUL6, M3K14_MOUSE | Q05512, MARK2_MOUSE | P53779, MK10_HUMAN |
| Q13233, M3K1_HUMAN | O08679, MARK2_RAT | Q61831, MK1_MOUSE |
| P53349, M3K1_MOUSE | P27448, MARK3_HUMAN | P49187, MK10_RAT |
| Q62925, M3K1_RAT | Q03141, MARK3_MOUSE | Q15759, MK11_HUMAN; |
| Q9Y2U5, M3K2_HUMAN | Q96L34,MARK4_HUMAN | Q9WUI1, MK11_MOUSE |
| Q61083, M3K2_MOUSE | Q9Y2H9, MAST1_HUMAN | O42376, MK12_BRARE |
| Q99759, M3K3_HUMAN | Q9R1L5, MAST1_MOUSE | P53778, MK12_HUMAN |
| Q61084, M3K3_MOUSE | Q810W7, MAST1_RAT | O08911, MK12_MOUSE |
| Q9Y6R4, M3K4_HUMAN | Q6P0Q8,MAST2_HUMAN | Q63538, MK12_RAT |
| O08648, M3K4_MOUSE | Q60592, MAST2_MOUSE | O15264, MK13_HUMAN |
| Q99683, M3K5_HUMAN | Q96GX5, MASTL_HUMA | Q9Z1B7, MK13_MOUSE |
| O35099, M3K5_MOUSE | Q8C0P0, MASTL_MOUSE | Q9N272, MK13_PANTR |
| O95382, M3K6_HUMAN | P38615, MDS1_YEAST | Q9WTY9, MK13_RAT |
| Q9V3Q6, M3K7_DROME | P38111, MEC1_YEAST | Q9DGE2, MK14A_BRARE |
| O43318, M3K7_HUMAN | Q10292, MEK1_SCHPO | Q90336, MK14A_CYPCA |
| Q62073, M3K7_MOUSE | P24719, MEK1_YEAST | O62618, MK14A_DROME |
| P41279, M3K8_HUMAN | Q14680, MELK_HUMAN | Q9DGE1, MK14B_BRARE |
| Q07174, M3K8_MOUSE | Q61846, MELK_MOUSE | Q9I958, MK14B_CYPCA |
| Q63562, M3K8_RAT | P43294, MHK_ARATH | O61443, MK14B_DROME |
| P80192, M3K9_HUMAN | Q23356, MIG15_CAEEL | P83100, MK14C_DROME |
| Q5TCX8, M3KL4_HUMAN | P00531, MIL_AVIMH | O02812, MK14_CANFA |
| Q95UN8, M3KSL_DROME | P46196, MK01_BOVIN | Q16539, MK14_HUMAN |
| P83104, M3LK7_DROME | P28482, MK01_HUMAN | P47811, MK14_MOUSE |
| Q92918, M4K1_HUMAN | P63085, MK01_MOUSE | Q95NE7, MK14_PANTR |
| P70218, M4K1_MOUSE | P63086, MK01_RAT | P70618, MK14_RAT |
| Q12851, M4K2_HUMAN | P26696, MK01_XENLA | P47812, MK14_XENLA |
| Q61161, M4K2_MOUSE | P27361, MK03_HUMAN | P43068, MKC1_CANAL |
| Q8IVH8, M4K3_HUMAN | Q63844, MK03_MOUSE | Q9BUB5, MKNK1_HUMA |
| Q99JP0, M4K3_MOUSE | P21708, MK03_RAT | O08605, MKNK1_MOUSE |
| Q924I2, M4K3_RAT | P31152, MK04_HUMAN | Q9HBH9, MKNK2_HUMAN |
| O95819, M4K4_HUMAN | Q6PSG0, MK04_MOUSE | Q8CDB0, MKNK2_MOU |
| P97820, M4K4_MOUSE | Q63454, MK04_RAT | Q9NYL2, MLTK_HUMAN |
| Q9Y4K4, M4K5_HUMAN | Q16659, MK06_HUMAN | Q9ESL4, MLTK_MOUSE |
| Q8BPM2, M4K5_MOUSE | Q61532, MK06_MOUSE | Q07176, MMK1_MEDSA |
| Q8N4C8, M4K6_HUMAN | P27704, MK06_RAT | Q40353, MMK2_MEDSA |
| Q9JM52, M4K6_MOUSE | Q13164, MK07_HUMAN | P49657, MNB_DROME |
| P20794, MAK_HUMAN | Q9WVS8, MK07_MOUSE | Q9UQ07, MOK_HUMAN |
| Q04859, MAK_MOUSE | Q90327, MK08A_CYPCA | Q9WVS4, MOK_MOUSE |
| P20793, MAK_RAT | O42099, MK08B_CYPCA | P87347, MOS_APTAU |
| Q8IW41, MAPK5_HUMAN | Q9DGD9, MK08_BRARE | Q8QHF0, MOS_ATHNI |
| O54992, MAPKS_MOUSE | P45983, MK08_HUMAN | Q8AX02, MOS_ATHSQ |
| Q00859, MAPK_FUSSO | Q91Y86, MK08_MOUSE | P10650, MOS_CERAE |
| Q06060, MAPK_PEA | P49185, MK08_RAT | P10741, MOS_CHICK |
| Q40884, MAPK_PETHY | Q8QHK8, MK08_XENLA | Q90XV8, MOS_CICNG |
| Q9P0L2, MARK1_HUMAN | P79996, MK09_CHICK | Q8AX01, MOS_DENAN |
| Q8VHJ5, MARK1_MOUSE | P45984, MK09_HUMAN | Q90XV6, MOS_GYMCA |
| O08678, MARK1_RAT | Q9WTU6, MK09_MOUSE | P00540, MOS_HUMAN |
| P00536, MOS_MOUSE | Q8K1R7, NEK9_MOUSE | Q9Z2A0, PDPK1_MOUSE |
| P07331, MOS_MSVMH | Q7ZZC8, NEK9_XENLA | O55173, PDPK1_RAT |
| P00537, MOS_MSVMM | P48479, NIM1_NEUCR | O74456, PEF1_SCHPO |
| P00538, MOS_MSVMO | P10676, NINAC_DROME | O94921, PFTK1_HUMAN |
| P32593, MOS_MSVMT | Q9UBE8, NLK_HUMAN | O35495, PFTK1_MOUSE |
| P10421, MOS_MSVTS | O54949, NLK_MOUSE | Q751E8, PHO85_ASHGO |
| Q90XV9, MOS_NYCNY | O48963, NPH1_ARATH | Q9HGY5, PHO85_CANAL |
| P50118, MOS_PIG | O42626, NRC2_NEUCR | Q6FKD4, PHO85_CANGA |
| P00539, MOS_RAT | Q08942, NRKA_TRYBB | Q6BRY2, PHO85_DEBHA |
| Q8AX00, MOS_SIBNE | Q03428, NRKB_TRYBB | Q92241, PHO85_KLULA |
| Q90XV7, MOS_VULGR | Q40517, NTF3_TOBAC | Q6C7U8, PHO85_YARLI |
| P12965, MOS_XENLA | Q40532, NTF4_TOBAC | P17157, PHO85_YEAST |
| P45985, MP2K4_HUMAN | Q40531, NTF6_TOBAC | Q9N0P9, PIM1_BOVIN |
| P47809, MP2K4_MOUSE | O60285, NUAK1_HUMAN | Q9YHZ5, PIM1_BRARE |
| O94235, MPH1_SCHPO | O13310, ORB6_SCHPO | Q95110, PIM1_FELCA |
| Q39021, MPK1_ARATH | Q17850, PAK1_CAEEL | P11309, PIM1_HUMAN |
| Q39022, MPK2_ARATH | Q13153, PAK1_HUMAN | P06803, PIM1_MOUSE |
| Q39023, MPK3_ARATH | O88643, PAK1_MOUSE | P26794, PIM1_RAT |
| Q39024, MPK4_ARATH | P35465, PAK1_RAT | Q9P1W9, PIM2_HUMAN |
| Q39025, MPKS_ARATH | P38990, PAK1_YEAST | Q62070, PIM2_MOUSE |
| Q39026, MPK6_ARATH | Q13177, PAK2_HUMAN | Q9PU85, PIM3_COTJA |
| Q39027, MPK7_ARATH | Q8CIN4, PAK2_MOUSE | Q86V86, PIM3_HUMAN |
| Q8AYG3, MPS1_BRARE | Q29502, PAK2_RABIT | P58750, PIM3_MOUSE |
| P54199, MPS1_YEAST | Q64303, PAK2_RAT | O70444, PIM3_RAT |
| P50873, MRK1_YEAST | O75914, PAK3_HUMAN | Q91822, PIM3_XENLA |
| Q8NEV4, MYO3A_HUMA | Q61036, PAK3_MOUSE | Q9BXM7, PINK1_HUMAN |
| Q8K3H5, MYO3A_MOUSE | Q7YQL4, PAK3_PANTR | Q99MQ3, PINK1_MOUSE |
| Q8WXR4, MY03B_HUMA | Q7YQL3, PAK3_PONPY | P42493, PK1_ASFB7 |
| O75011, NAK1_SCHPO | Q62829, PAK3_RAT | P34206, PK1_ASFM2 |
| P43293, NAK_ARATH | O96013, PAK4_HUMAN | P41415, PK1_NPVAC |
| P84199, NEK1_CAEEL | Q8BTW9, PAK4_MOUSE | P41719, PK1_NPVHZ |
| Q96PY6, NEK1_HUMAN | Q9NQU5, PAK6_HUMAN | P41720, PK1_NPVLD |
| P51954, NEK1_MOUSE | Q9P286, PAK7_HUMAN | O10269, PK1_NPVOP |
| P51955, NEK2_HUMAN | Q9VXE5, PAKM_DROME | Q9KIG4, PK1_STRTO |
| O35942, NEK2_MOUSE | Q96RG2, PASK_HUMAN | P41676, PK2_NPVAC |
| P51956, NEK3_HUMAN | Q8CEE6, PASK_MOUSE | Q9W0V1, PK61C_DROME |
| Q9R0A5, NEK3_MOUSE | Q9FE20, PBS1_ARATH | P54739, PKAA_STRCO |
| P51957, NEK4_HUMAN | Q00536, PCTK1_HUMAN | P54740, PKAB_STRCO |
| Q9Z1J2, NEK4_MOUSE | Q04735, PCTK1_MOUSE | Q03407, PKH1_YEAST |
| Q9HC98, NEK6_HUMAN | Q63686, PCTK1_RAT | Q16512, PKIA_HUMAN |
| Q9ES70, NEK6_MOUSE | Q00537, PCTK2_HUMAN | P70268, PKL1_MOUSE |
| P59895, NEK6_RAT | Q8K0D0, PCTK2_MOUSE | Q63433, PKL1_RAT |
| Q8TDX7, NEK7_HUMAN | O35831, PCTK2_RAT | Q16513, PKL2_HUMAN |
| Q9ES74, NEK7_MOUSE | Q07002, PCTK3_HUMAN | Q8BWW9, PKL2_MOUSE |
| Q90XC2, NEK8_BRARE | Q04899, PCTK3_MOUSE | O08874, PKL2_RAT |
| Q86SG6, NEK8_HUMAN | Q5RD01, PCTK3_PONPY | P37562, PKN1_BACSU |
| Q91ZR4, NEK8_MOUSE | O35832, PCTK3_RAT | Q822R1, PKN1_CHLCV |
| Q8TD19, NEK9_HUMAN | O15530, PDPK1_HUMAN | Q9PKP3, PKN1_CHLMU |
| Q7AJA5, PKN1_CHLPN | P75524, PKNS_MYCPN | P47116, PTK2_YEAST |
| O84147, PKN1_CHLTR | Q9XA16, PKNX_STRCO | Q9FKS4, RAD3A_ARATH |
| Q8FUI5, PKN1_COREF | Q01577, PKPA_PHYBL | Q02099, RAD3_SCHPO |
| Q8NU98, PKN1_CORGL | Q9S2C0, PKSC_STRCO | P22216, RAD53_YEAST |
| P33973, PKN1_MYXXA | P49695, PKWA_THECU | P05625, RAF1_CHICK |
| Q8R9T6, PKN1_THETN | P34331, PLK1_CAEEL | P04049, RAF1_HUMAN |
| O34507, PKN2_BACSU | P53350, PLK1_HUMAN | Q99N57, RAF1_MOUSE |
| Q97IC2, PKN2_CLOAB | Q07832, PLK1_MOUSE | P11345, RAF1_RAT |
| Q8XJL8, PKN2_CLOPE | Q62673, PLK1_RAT | P09560, RAF1_XENLA |
| Q8FUI4, PKN2_COREF | P70032, PLK1_XENLA | P00532, RAF_MSV36 |
| Q8NU97, PKN2_CORGL | P62205, PLKLXENTR | P38622, RCK1_YEAST |
| P54736, PKN2_MYXXA | Q9N2L7, PLK2_CAEEL | P38623, RCK2_YEAST |
| Q9XBQ0, PKN3_MYXXA | Q9NYY3, PLK2_HUMAN | P43565, R1M15_YEAST |
| P54737, PKN5_MYXXA | P53351, PLK2_MOUSE | Q12196, RIO1_YEAST |
| P54738, PKN6_MYXXA | Q9R012, PLK2_RAT | P40160, RIO2_YEAST |
| Q8G4G1, PKNA2_BIFLO | Q20845, PLK3_CAEEL | Q9BRS2, RIOK1_HUMAN |
| P54734, PKNA_ANASP | Q9H4B4, PLK3_HUMAN | Q9BVS4, RIOK2_HUMAN |
| P65727, PKNA_MYCBO | Q60806, PLK3_MOUSE | Q9CQS5, RIOK2_MOUSE |
| P54743, PKNA_MYCLE | Q9R011, PLK3_RAT | O14730, RIOK3_HUMAN |
| P65726, PKNA_MYCTU | O00444, PLK4_HUMAN | Q9DBU3, RIOK3_MOUSE |
| Q8G6P9, PKNB_BIFLO | Q64702, PLK4_MOUSE | Q13546, RIPK1_HUMAN |
| Q9CEF5, PKNB_LACLA | P50528, PLO1_SCHPO | Q60855, R1PK1_MOUSE |
| P0A5S5, PKNB_MYCBO | Q17446, PMK1_CAEEL | O43353, R1PK2_HUMAN |
| P54744, PKNB_MYCLE | Q8MXI4, PMK2_CAEEL | P58801, R1PK2_MOUSE |
| P0A5S4, PKNB_MYCTU | O44514, PMK3_CAEEL | Q9Y572, RIPK3_HUMAN |
| Q822K5, PKND_CHLCV; | O18209, PMYT1_CAEEL | Q9QZL0, RIPK3_MOUSE |
| Q9PK92, PKND_CHLMU | Q9NI63, PMYT1_DROME | Q9Z2P5, RIPK3_RAT |
| Q9Z986, PKND_CHLPN | Q99640, PMYT1_HUMAN | P57078, R1PK4_HUMAN |
| O84303, PKND_CHLTR | Q9ESG9, PMYT1_MOUSE | Q9LQQ8, RLCK7_ARATH |
| O05871, PKND_MYCTU | Q91618, PMYT1_XENLA | P47735, RLK5_ARATH |
| Q7TZN3, PKNE_MYCBO | P52304, POLO_DROME | P27966, RMIL_AVEVR |
| P72001, PKNE_MYCTU | Q09690, POM1_SCHPO | P10533, RMIL_AVII1 |
| Q7TZN1, PKNF_MYCBO | O13958, PRK1_SCHPO | Q8MIT6, ROCK1_BOVIN |
| P72003, PKNF_MYCTU | P78527, PRKDC_HUMAN | Q13464, ROCK1_HUMAN |
| P65729, PKNG_MYCBO | P97313, PRKDC_MOUSE | P70335, ROCK1_MOUSE |
| P57993, PKNG_MYCLE | P51817, PRKX_HUMAN | P61584, ROCK1_PANTR |
| P65728, PKNG_MYCTU | Q922R0, PRKX_MOUSE | O77819, ROCK1_RABIT |
| Q7U095, PKNH_MYCBO | O43930, PRKY_HUMAN | Q63644, ROCK1_RAT |
| Q11053, PKNH_MYCTU | Q13523, PRP4B_HUMAN | Q28021, ROCK2_BOVIN |
| P65731, PKNI_MYCBO | Q61136, PRP4B_MOUSE | O75116, ROCK2_HUMAN |
| P65730, PKNI_MYCTU | Q07538, PRP4_SCHPO | P70336, ROCK2_MOUSE |
| P65733, PKNJ_MYCBO | Q96S44, PRPK_HUMAN | Q62868, ROCK2_RAT |
| P65732, PKNJ_MYCTU | Q99PW4, PRPK_MOUSE | P93194, RPK1_IPONI |
| Q7TXA9, PKNK_MYCBO | Q12706, PSK1_SCHPO | P42411, RSBT_BACSU |
| P95078, PKNK_MYCTU | Q9ZVR7, PSKR_ARATH | Q9K5J7, RSBW_BACAN |
| Q7TYY6, PKNL_MYCBO | Q8LPB4, PSKR_DAUCA | Q73CI0, RSBW_BACC1 |
| O53510, PKNL_MYCTU | P36002, PTK1_YEAST | Q81H23, RSBW_BACCR |
| P47355, PKNS_MYCGE | Q6FRE7, PTK2_CANGA | Q63F14, RSBW_BACCZ |
| Q9KFF1, RSBW_BACHD | Q09488, SMA6_CAEEL | Q9UEE5, ST17A_HUMAN |
| Q6HMH0, RSBW_BACHK | P41808, SMK1_YEAST | Q9GM70, ST17A_RABIT |
| O50231, RSBW_BACLI | P57059, SN1L1_HUMAN | O94768, ST17B_HUMAN |
| P17904, RSBW_BACSU | Q60670, SN1L1_MOUSE | Q8BG48, ST17B_MOUSE |
| Q92DC2, RSBW_LISIN | Q9R1U5, SN1L1_RAT | Q91XS8, ST17B_RAT |
| Q721S2, RSBW_LISMF | Q9IA88, SN1L2_CHICK | Q9Y2H1, ST38L_HUMAN |
| Q8Y8K6, RSBW_LISMO | Q9H0K1, SN1L2_HUMAN | Q7TSE6, ST38L_MOUSE |
| Q8CXL7, RSBW_OCEIH | Q8CFH6, SN1L2_MOUSE | P23561, STE11_YEAST |
| Q5HED6, RSBW_STAAC | Q5REX1, SN1L2_PONPY | Q92212, STE20_CANAL |
| P0A0H6, RSBW_STAAM | Q81MF4, SP2AB_BACAN | Q03497, STE20_YEAST |
| P0A0H7, RSBW_STAAN | Q731M3, SP2AB_BACC1 | P46599, STE7_CANAL |
| Q6GF08, RSBW_STAAR | P70878, SP2AB_BACCO | P06784, STE7_YEAST |
| Q6G7P4, RSBW_STAAS | Q819B3, SP2AB_BACCR | O94804, STK10_HUMAN |
| P0A0H8, RSBW_STAAU | Q635K7, SP2AB_BACCZ | O55098, STK10_MOUSE |
| Q8NVI5, RSBW_STAAW | Q9KCN2, SP2AB_BACHD | Q15831, STK11_HUMAN |
| Q9F7V2, RSBW_STAEP | Q6HE93, SP2AB_BACHK | Q91604, STK11_XENLA |
| Q75LR7, SAPK1_ORYSA | P26778, SP2AB_BACLI | O75716, STK16_HUMAN |
| Q84TC6, SAPK2_ORYSA | P35148, SP2AB_BACME | O88697, STK16_MOUSE |
| Q75V63, SAPK3_ORYSA | O32724, SP2AB_BACSH | P57760, STK16_RAT |
| Q5N942, SAPK4_ORYSA | Q5WH26, SP2AB_BACSK | P49842, STK19_HUMAN |
| Q7XKA8, SAPK5_ORYSA | O32727, SP2AB_BACST | Q9JHN8, STK19_MOUSE |
| Q6ZI44, SAPK6_ORYSA | P10728, SP2AB_BACSU | Q9UPE1, STK23_HUMAN |
| Q7XQP4, SAPK7_ORYSA | Q97GQ9, SP2AB_CLOAB | Q9Z0G2, STK23_MOUSE |
| Q7Y0B9, SAPK8_ORYSA | Q8XIR5, SP2AB_CLOPE | Q9Y6E0, STK24_HUMAN |
| Q75V57, SAPK9_ORYSA | P59623, SP2AB_CLOTE | Q99KH8, STK24_MOUSE |
| Q75H77, SAPKA_ORYSA | Q8EQ73, SP2AB_OCEIH | O00506, STK25_HUMAN |
| P25333, SAT4_YEAST | O32721, SP2AB_PAEPO | Q9Z2W1, STK25_MOUSE |
| P11792, SCH9_YEAST | P59624, SP2AB_PASPE | Q9BXU1, STK31_HUMAN |
| P50530, SCK1_SCHPO | Q8RAA8, SP2AB_THETN | Q99MW1, STK31_MOUSE |
| P18431, SGG_DROME | Q61IS6, SPK1_CAEBR | Q8TDR2, STK35_HUMAN |
| O00141, SGK1_HUMAN | Q03563, SPK1_CAEEL | Q15208, STK38_HUMAN |
| Q9WVC6, SGK1_MOUSE | P27638, SPK1_SCHPO | Q91VJ4, STK38_MOUSE |
| Q9XT18, SGK1_RABIT | Q9FAB3, SPKA_SYNY3 | Q9UEW8, STK39_HUMAN |
| Q06226, SGK1_RAT | P74297, SPKB_SYNY3 | Q9Z1W9, STK39_MOUSE |
| Q9HBY8, SGK2_HUMAN | P74745, SPKC_SYNY3 | O88506, STK39_RAT |
| Q9QZS5, SGK2_MOUSE | P54735, SPKD_SYNY3 | Q13188, STK3_HUMAN |
| Q8R4U9, SGK2_RAT | P73469, SPKF_SYNY3 | Q9JI10, STK3_MOUSE |
| Q96BR1, SGK3_HUMAN | Q92398, SPM1_SCHPO | Q13043, STK4_HUMAN |
| Q9ERE3, SGK3_MOUSE | Q9UQY9, SPO4_SCHPO | Q91819, STK6L_XENLA |
| P23293, SGV1_YEAST | Q96SB4, SPRK1_HUMAN | O14965, STK6_HUMAN |
| P50527, SHK1_SCHPO | O70551, SPRK1_MOUSE | P97477, STK6_MOUSE |
| Q10056, SHK2_SCHPO | Q5RD27, SPRK1_PONPY | P59241, STK6_RAT |
| O14305, SID1_SCHPO | P78362, SPRK2_HUMAN | Q91820, STK6_XENLA |
| Q09898, SID2_SCHPO | O94547, SRK1_SCHPO | P83098, STLK_DROME |
| Q12469, SKM1_YEAST | Q09092, SRK6_BRAOE | Q9S713, STT7_ARATH |
| Q12505, SKS1_YEAST | O54781, SRPK2_MOUSE | Q84V18, STT7_CHLRE |
| Q03656, SKY1_YEAST | P25390, SSK22_YEAST | Q09892, STY1_SCHPO |
| Q00772, SLT2_YEAST | P50526, SSP1_SCHPO | P46549, SULU_CAEEL |
| P39745, SUR1_CAEEL | Q9J5B1, V111_FOWPV | Q20085, YPS7_CAEEL |
| Q05913, T2FA_DROME | Q9J523, V212_FOWPV | Q09499, YQG4_CAEEL |
| P35269, T2FA_HUMAN | Q9J509, V226_FOWPV | Q09298, YQO9_CAEEL |
| Q04870, T2FA_XENLA | Q03785, VHS1_YEAST | Q20347, YR62_CAEEL |
| P41895, T2FA_YEAST | O57252, VPK1_VACCA | Q09595, YRL5_CAEEL |
| P51123, TAF1_DROME | P20505, VPK1_VACCC | Q11090, YWY3_CAEEL |
| P21675, TAF1_HUMAN | P16913, VPK1_VACCV | Q621J7, ZYG1_CAEBR |
| Q15569, TESK1_HUMAN | P33800, VPK1_VARV | Q9GT24, ZYG1_CAEEL |
| O70146, TESK1_MOUSE | P32216, VPK2_SWPVK | |
| Q63572, TESK1_RAT | O57177, VPK2_VACCA | |
| Q96S53, TESK2_HUMAN | P21095, VPK2_VACCC | |
| Q8VCT9, TESK2_MOUSE | P29884, VPK2_VACCP | |
| Q924U5, TESK2_RAT | Q9JFE5, VPK2_VACCT | |
| P36897, TGFR1_HUMAN | Q89121, VPK2_VACCV; | |
| Q64729, TGFR1_MOUSE | P33801, VPK2_VARV | |
| P80204, TGFRl_RAT | Q9UVG6, VPS15_PICPA | |
| P37173, TGFR2_HUMAN | P22219, VPS15_YEAST | |
| Q62312, TGFR2_MOUSE | Q7ZUS1, VRK1_BRARE | |
| P38551, TGFR2_PIG | Q99986, VRK1_HUMAN | |
| P38438, TGFR2_RAT | Q80X41, VRK1_MOUSE | |
| P34314, TLK1_CAEEL | Q86Y07, VRK2_HUMAN | |
| Q9UKI8, TLK1_HUMAN | Q8BN21, VRK2_MOUSE | |
| Q8C0V0, TLK1_MOUSE | Q8IV63, VRK3_HUMAN | |
| Q86UE8, TLK2_HUMAN | Q8K3G5, VRK3_MOUSE | |
| O55047, TLK2_MOUSE | Q9H4A3, WNK1_HUMAN | |
| Q9UKE5, TNIK_HUMAN | P83741, WNK1_MOUSE | |
| P83510, TNIK_MOUSE | Q9JIH7, WNK1_RAT | |
| Q6DHU8, TOPK_BRARE | Q9Y3S1, WNK2_HUMAN | |
| Q96KB5, TOPK_HUMAN | Q9BYP7, WNK3_HUMAN | |
| Q9JJ78, TOPK_MOUSE | Q96J92, WNK4_HUMAN | |
| Q9BX84, TRPM6_HUMAN | Q80UE6, WNK4_MOUSE | |
| Q8CIR4, TRPM6_MOUSE | Q7TPK6, WNK4_RAT | |
| Q96QT4,TRPM7_HUMAN | Q58473, Y1073_METJA | |
| Q923J1, TRPM7_MOUSE | Q8MYQ1, Y31E_CAEEL | |
| Q9BXA7,TSSK1_HUMAN | Q03021, Y396_THEAC | |
| Q61241, TSSK1_MOUSE | Q57886, Y444_METJA | |
| Q96PF2, TSSK2_HUMAN | P34516, YMX8_CAEEL | |
| O54863, TSSK2_MOUSE | P45894, YNA3_CAEEL | |
| Q96PN8, TSSK3_HUMAN | P32742, YNH4_CAEEL | |
| Q9D2E1, TSSK3_MOUSE | P34633, YOO1_CAEEL | |
| Q6SA08, TSSK4_HUMAN | P34635, YOO3_CAEEL | |
| Q9D411, TSSK4_MOUSE | P34649, YOT3_CAEEL | |
| Q8TAS1, UHMK1_HUMA | Q09437, YP62_CAEEL | |
| P97343, UHMK1_MOUSE | Q11179, YPC2_CAEEL | |
| Q63285, UHMK1_RAT | P12688, YPK1_YEAST | |
| O75385, ULK1_HUMAN | P18961, YPK2_YEAST | |
| O70405, ULK1_MOUSE | Q9RI12, YPKA_YERPE | |
| Q23023, UNC51_CAEEL | Q05608, YPKA_YERPS | |

Analysis of each one of these enzymes, alone or in combination with others, is specifically contemplated in accordance with the teachings herein, as part of the invention.

Kinases associated with cancers include at least the following: Ab1 and BCR (BCR-Ab1 fusion, chronic myelogenous leukemia); Agc (within PI3-kinase signaling pathway; over-expressed in breast, prostate, lung, pancreatic, liver, ovarian, and colorectal cancers); Akt2 (amplified and over-expressed in ovarian and pancreatic tumors); Alk (lymphomas); Arg (differential expression in multiple cancers); Atm (loss-of-function mutations correlate with leukemias and lymphomas); Atr (stomach, endometrial cancers); AurA and AurB (amplified or overexpressed in many tumors); Axl (overexpressed in many cancers); B-Raf (melanoma and other cancers); Brk (breast and other cancers); BUB1 and BUBR1 (gastric and other cancers); Cdk1, Cdk2, Cdk4, and Cdk6 (activated in many cancers); Ck2 (lung and breast cancers); Cot/Tp12 (overexpressed in breast tumors); Ctk/MatK (breast cancer); DapK1; eEG2k (breast cancer); EGFR (over-expressed in head & neck and breast cancers); EphA1, EphA2, EphA3, EphB2, and EphB4 (multiple cancers); Fak (breast, ovarian, thyroid, other cancers); Fer (prostate); FGFR-1, FGFR-2, FGFR-3, and FGFR-4 (numerous cancers); Fgr (prostate); VEGFR-1, VEGFR-2, and VEGFR-3 (numerous cancers); mTOR (numerous cancers); FMS (breast and other cancers); Her-2, Her-3, and Her-4 (breast and other cancers); Hgk; HipK1 and HipK2; Ilk (increased expression in multiple tumors); Jak-1 and Jak-2; Kit (gastrointestinal stromal tumors); Lck (overexpressed in thymic tumors and other cancers); Met (numerous cancers); Mst4 (prostate cancer); NEK2 and NEK8; p38; Pak4 (overexpressed in several cancers); PDGFR-α and β; Pim1 (overexpressed in prostate cancer); Pim2 and Pim3; Pkc-α, Pkc-β, Pkc-δ, Pkc-ε, Pkc-η, and Pkc-θ (numerous cancers); Pkr; Plk1 (elevated expression in many cancers); Raf1 (amplified in many tumors); Ret; Ron (highly expressed in numerous cancers); p70s6k (elevated expression in colon and breast cancer); Src (increased expression and activity in numerous cancers); Syk (reduced expression in numerous cancers); TGFβR-1 and TGFβR-2; Tie2; TrkB; Tyro3; and Yes (amplification and/or increased expression in multiple cancers).

Kinases associated with cardiovascular disease or hypertension include A1k1, NPR1, BMPR2, CDK9, Erk5, Pkc-α, Pkc-δ, Pkc-ε, ROCK1 and ROCK 2, Tie 2, and Wnk1 and Wnk4.

Kinases associated with neurodegeneration, neurological, or central nervous system diseases include ATM (loss of function mutations associated with ataxia); CK1α, CK1δ, CK2α1 and CK2α2; DAPK1 (increased expression in epilepsy); DMPK1; Dyrk1a; Fyn (epilepsy); Gsk3α and GSK3β; Jnk3; Pak2; Pink1 (Parkinson's disease); PKcε (Alzheimer's disease); Pkcγ; Pkr; ROCK1 (Alzheimer's disease); and Rsk2.

The CDK9 kinase is associated with viral infection and replication, and inhibitors have been shown to block HIV replication and varicella zoster replication. Blockage of MEK1 and MEK2 appears to block export of influenza viral particles.

The Flt4 receptor tyrosine kinase (VEGFR-3) has been associated with lymphangiogenesis and loss of function mutations associated with lymphedema.

Loss of function mutations in JAK3 are associated with severe combined immunodeficiency (SCID).

The enzymes that are evaluated using the disclosed methods may be involved in a signaling pathway. Signaling pathways include PI3K/AKT pathways; Ras/Raf/MEK/Erk pathways; MAP kinase pathways; JAK/STAT pathways; mTOR/TSC pathways; heterotrimeric G protein pathways; PKA pathways; PLC/PKC pathways; NK-kappaB pathways; cell cycle pathways (cell cycle kinases); TGF-beta pathways; TLR pathways; Notch pathways; Wnt pathways; Nutrient signaling pathways (AMPK signaling); cell-cell and cell:substratum adhesion pathways (such as cadherin, integrins); stress signaling pathways (high/low salt, heat, radiation); cytokine signaling pathways; antigen receptor signaling pathways; and co-stimulatory immune signaling pathways. In some cases, the methods may be used to measure the activity of more than one enzyme involved in the same signaling pathway. Numerous resources are widely known with descriptions of pathways, including www.biocarta.com, www.cellsignal.com, and www.signaling-gateway.org.

The term “specific for” with respect to a substrate and enzyme in a sample refers to a substrate that has a reactive site or “domain” which an enzyme recognizes and modifies or catalyzes the modification of, and which is not known to be modified at the same site/domain in the same way by another active enzyme in the sample. In cases where all enzymes in a sample are known, the substrate is specific for one enzyme when no other enzyme in the sample modifies it at the same site. In a cell lysate or enzyme mixture containing an indeterminate population of enzymes, specificity is maximized based on knowledge available and can be enhanced with the addition of inhibitor substances that inhibit enzymes other than the enzyme of interest. In various cases, the substrate is a polypeptide that comprises the complete amino acid sequence of the naturally occurring protein, while in other cases, the substrate is a polypeptide that comprises the amino acid sequence of the reactive site or domain for the enzyme. The domain of the substrate can be a reactive site and/or a biologically relevant domain of the substrate, such as an extracellular domain, intracellular domain, an independently folding portion, or a functional domain.

Table 3, below, shows some substrates that are associated with various kinases and the position of their modification by the kinase.

TABLE 3

| Protein Substrate | Site | Upstream Kinase |
|---|---|---|
| β catenin | S33 | GSK |
| | S37 | GSK |
| | T41 | GSK |
| | S45 | CK1 |
| | Y86 | Src |
| | T102 | CK2 |
| | T112 | CK2 |
| | Y142 | Fyn |
| 4EBP1 | S7 | ATM |
| | T36 | mTOR |
| | T45 | mTOR |
| | S64 | mTOR |
| | S65 | MAPK |
| | T69 | mTOR |
| | S82 | mTOR |
| | S100 | mTOR |
| | S111 | ATM |
| S6 | S235 | S6K |
| | S236 | S6K |
| | S240 | Unknown |
| | S242 | Unknown |
| | S244 | Unknown |
| | S257 | Unknown |
| cMyc | T8 | RAF |
| | T58 | GSK |
| | S62 | MAPK1 |
| SMAD1 | S187 | MAPK1 |
| | S195 | MAPK1 |
| | S206 | MAPK1 |
| | S214 | MAPK1 |
| | S462 | TGFbR1 |
| | S463 | TGFbR1 |
| | S465 | TGFbR1 |
| BAD | Ser 75 | PKA, MAPK, RSK1 |
| | S99 | PKB |
| | S118 | RSK1 |
| FOXO3 | Thr32 | PKB, SGK |
| | Ser253 | PKB, SGK |
| | Ser253 | STK4 |
| | Ser315 | SGK |
| | S318 | Unknown |
| | S321 | Unknown |
| | S644 | IKKBETA |
| STAT3 | S727 | Unknown |
| | Y691 | JAK kinases |
| | Y705 | JAK kinases |
| Annexin A1 | S4 | unknown |
| | Y20 | EGFR, Abl, Scr |
| | T23 | PKC |
| | S26 | PKC |
| | T215 | PKA |
| | Y207 | unknown |
| P53 | S9 | unknown |
| | S15 | ATM, DNA-PK |
| | T18 | CK1 |
| | S20 | CHK2 |
| | S33 | GSK3, CDK |
| | S37 | DNAPK |
| | S46 | HIPK2 |
| | T55 | MAPK |
| | S215 | AUROR A A |
| | S315 | AUROR A A, CDK |
| | S371 | CDK7 |
| | S376 | CDK7 |
| | S378 | CDK7 |
| | S392 | CK2, CDK7, EIF2AK2 |

In some cases, the substrate can be modified by more than one enzyme. For example, a substrate can have a second reactive site, or “domain,” which a second enzyme recognizes and modifies. In such instances, the substrate can be specific for the first enzyme by addition of an inhibitor of the second enzyme or the substrate can be specific for both enzymes at the same time. A particular substrate may be a wild-type substrate for two or more enzymes. Alternatively, a synthetic substrate can be engineered (e.g., as a fusion protein) to contain sites recognized by two or more enzymes. In various cases, the substrate can include at least one protein affinity tag. This tag can be used in an optional purification step after modification of the substrate by the enzyme, either before or after cleavage into fragments. Protein affinity tags contemplated include a His tag, a glutathione-S-transferase tag, a strepavidin tag, a thioredoxin tag, a c-myc tag, a calmodulin tag, a FLAG-tag, a maltose-binding protein tag, a Nus tag, a TAP-tag, or combinations thereof. Any purification technique can be used that is useful for chemical or biochemical separation, including the use of chromatographic techniques, affinity purification materials and methods, electrophoresis techniques, and the like. In certain cases, the purification is done by high pressure liquid chromatography (HPLC).

In some embodiments, the methods described herein are directed toward analysis of one or more enzyme activities in a sample. The term, a “plurality of enzymes” refers to at least two enzymes and embraces 3, 4, 5, 6, 7, 8, 9, 10, 20, 50, 100, or more enzymes. In some cases, samples for use in the disclosed methods can be any sample that contains an enzyme which catalyzes a reaction wherein the substrate and/or product of that reaction is/are not amenable to detection by mass spectrometry (MS). Substrates and products not amenable to detection by MS, as used herein, are entities that have a molecular weight greater than the detection range of a MS instrument. In various cases, the molecular weight of the substrate and/or product is greater than about 5 kDa, greater than about 6 kDa, greater than about 7 kDa, greater than about 8 kDa, greater than about 9 kDa, greater than about 10 kDa, greater than about 11 kDa, greater than about 12 kDa, greater than about 13 kDa, greater than about 14 kDa, or greater than about 15 kDa, 20 kDa, 25 kDa, 30 kDa, 40 kDa, 50 kDa, 75 kDa, 100 kDa, 125 kDa, 150 kDa, or 200 kDa. In some specific embodiments, the molecular weight of the substrate is about 7 to about 10 kDa. In one embodiment, substrates and/or products are proteins.

The sample that contains the enzyme for assay may be from any source, including any organism. Exemplary organisms are prokaryotes, eukaryotes, protists, fungii, plants, animals including humans or other mammals, and may be either crude or purified. In some embodiments, the sample is from a human or animal subject that is suspected of suffering from a disease characterized by changed in activity of one or more enzymes involved in a cellular process. Crude samples are samples that have not undergone significant purification prior to analysis, such as gel electrophoresis or other types of purification (e.g., liquid chromatography, size exclusion chromatography, and the like). Purified samples may be samples of individually purified enzymes or samples of mixture of enzymes purified prior to sample preparation. Samples may be cell lysates, whole cell samples, biopsy samples, and the like. In some variations, the sample is snap frozen (frozen using dry ice or liquid nitrogen) after collection and kept at a temperature below −40° C. prior to analysis. The sample may be a bodily fluid, secretion, or excretion, including, but not limited to, whole blood, serum, plasma, urine, feces, semen, mucus, saliva, tears, sweat, or gastric fluids. The samples may contain more than one enzyme, and the methods may be used to detect simultaneously the activity of more than one enzyme present in the sample. In some cases, the enzyme in the sample may be immunopurified, to produce a crude purified enzyme fraction, prior to analysis. This step can be performed for any enzyme and is especially useful in cases where the substrates for the target enzyme do not show the desired specificity, or when the aim is to determine the activity of enzyme isoforms showing the same substrate specificity.

When the sample being analyzed comes from an animal or human tissue sample, the sample can optionally be prepared in the following manner. The tissue is placed in a homogenizer (e.g., Dounce, Potter-Elvehjem or Eppendorf homogenizers) containing an appropriate lysis buffer. A non-limiting example of a suitable lysis buffer is 40 mM Hepes pH 7.5, 120 mM NaCl, 1 mM EDTA, 10 mM pyrophosphate, 10 mM glycerophosphate, 50 mM NaF, 0.5 mM orthovanadate, 0.3% CHAPS, and complete protease inhibitors (such as those provided by Roche). The tissue is then homogenized by several strokes with the pestle. The resulting homogenate s then centrifuged (e.g., 13,000×g for 15 minutes) to pellet insoluble material. The supernatant can then be used as enzyme source/sample for the multiplex kinase assays described herein.

The methods described herein can be used to compare kinase activity profiles between two different samples. For example, samples taken from the same source (e.g., the same patient) can be analyzed to assess kinase activity changes over time. Additionally and alternatively, kinase activity of a sample from a healthy source can be compared kinase activity from a challenged source (e.g., a health cell line, a healthy patient vs. a cancer cell line or a patient suspected of having or diagnosed with cancer).

Biological samples may be concentrated or diluted prior to analysis, depending on the concentration or activity of enzyme that is expected to be present in the sample. Because the methods described herein measure enzymatic activity by detection of products of the enzymatic reaction, small amounts of enzyme present can be detected simply by allowing the enzymatic reaction to proceed for long periods of time, to convert more substrate into product. The amplification effect of the methods disclosed herein, therefore, allow for highly sensitive means of evaluating enzyme activity. Very little sample is needed for meaningful analysis. In some cases, the sample may be a cell lysate of 100 cells or less, or 25 cells or less, or 10 cells or less, or one cell or less.

The term “particle” refers to any compound or substance with a capacity to have a substrate attached to its surface and susceptible to rapid separation from an enzymatic reaction mixture using techniques such as centrifugation, magnetic separation, size filtration, and other convention laboratory techniques. Particles can include for example and without limitation, a metal, a semiconductor, and an insulator particle compositions, and a dendrimer (organic or inorganic). Thus, particles are contemplated for use in the methods which comprise a variety of inorganic materials including, but not limited to, metals, semi-conductor materials or ceramics as described in U.S. Patent Publication No 20030147966. Ceramic particle materials include, but are not limited to, brushite, tricalcium phosphate, alumina, silica, and zirconia. Organic materials from which particles are produced include carbon. Particles as disclosed herein can be one or more polymers. Specific polymers contemplated include polystyrene, silicone rubber, polycarbonate, polyurethanes, polypropylenes, polymethylmethacrylate, polyvinyl chloride, polyesters, polyethers, and polyethylene. Biodegradable, biopolymer (e.g. polypeptides such as BSA, polysaccharides, etc.), other biological materials (e.g. carbohydrates), and/or polymeric compounds are also contemplated for use in the disclosed particles.

The particle can be metallic, or a colloidal metal. Thus, in various embodiments, particles useful in the practice of the methods include metal (including for example and without limitation, gold, silver, platinum, aluminum, palladium, copper, cobalt, indium, nickel, or any other metal amenable to nanoparticle formation), semiconductor (including for example and without limitation, CdSe, CdS, and CdS or CdSe coated with ZnS) and magnetic (for example, ferromagnetite) colloidal materials. Other particles useful in the practice of the invention include, also without limitation, ZnS, ZnO, Ti, TiO₂, Sn, SnO₂, Si, SiO₂, Fe, Fe⁺⁴, Ag, Cu, Ni, Al, steel, cobalt-chrome alloys, Cd, titanium alloys, AgI, AgBr, HgI₂, PbS, PbSe, ZnTe, CdTe, In₂S₃, In₂Se₃, Cd₃P₂, Cd₃As₂, InAs, and GaAs. Methods of making ZnS, ZnO, TiO₂, AgI, AgBr, HgI₂, PbS, PbSe, ZnTe, CdTe, In₂S₃, In₂Se₃, Cd₃P₂, Cd₃As₂, InAs, and GaAs particles are also known in the art. See, e.g., Weller, Angew. Chem. Int. Ed. Engl., 32, 41 (1993); Henglein, Top. Curr. Chem., 143, 113 (1988); Henglein, Chem. Rev., 89, 1861 (1989); Brus, Appl. Phys. A., 53, 465 (1991); Bahncmann, in Photochemical Conversion and Storage of Solar Energy (eds. Pelizetti and Schiavello 1991), page 251; Wang and Herron, J. Phys. Chem., 95, 525 (1991); Olshaysky, et al., J. Am. Chem. Soc., 112, 9438 (1990); Ushida et al., J. Phys. Chem., 95, 5382 (1992). In some specific embodiments, the particle comprises cross-linked agarose (sold under the trade name SEPHAROSE®) or silica. In various embodiments, the particles are magnetic.

In preferred embodiments, the particle does not aggregate on the bottom of the reaction vessel. Avoidance of this aggregation can be performed by using particles with a higher density than the reaction medium and a stir bar or some other agitation device to keep the particles mixing with the reaction medium. Additionally and alternatively, the particles can be of a density that allows the particle to be suspended within the reaction medium (e.g., the aqueous, lipid, or other formulation suitable for the enzyme of interest) and not settle to the bottom of the reaction vessel within time periods required for the enzymatic reaction (usually seconds, minutes, or hours). In certain embodiments, both agitation and lower density particles are employed. In some embodiments, lower density particles that do not aggregate on the bottom of a reaction vessel are particles having a diameter less than about 10 μm, less than about 5 μm, less than about 4 μm, less than about 3 μm, or less than about 2 μm.

The terms “associated with” or “attached to,” as used herein, refer to an interaction between the surface of the particle and the substrate and/or product. That interaction can be through any means. Regardless of the means by which the substrate and/or product is attached to or associated with the particle, in embodiments where the substrate is a protein, attachment in various aspects is effected through a N-terminal or C-terminal linkage, some type of internal linkage (e.g., amino acid side-chain or disulfide linkage), or any combination of these attachments. In some embodiments, the association is via a covalent interaction. Other means of association are also contemplated, such as ionic interaction, van der Waals interactions, hydrophobic interactions, and mixtures of such interactions. In some embodiments, a protein or other enzyme substrate can be modified with a linker moiety that allows for attachment to a particle surface. In various embodiments, side chains of amino acids can be used to attach a substrate to the particle surface. Such amino acids contemplated include lysine, ornithine, glutamic acid, aspartic acid, cysteine, serine, and threonine. In embodiments where the substrate is not a protein, the substrate can be modified to include an amino acid and the amino acid can be attached to the surface of the particle. Immobilization of molecules in general is described in, e.g., U.S. Pat. No. 6,465,178; U.S. Patent Publication No. 2008/0090306; and 2008/0102036. Amination, hydroxylation, carboxylation, etc. of a surface (such as a polymeric surface) can be accomplished using corona discharge or a plasma glow discharge. Such methods are disclosed in, for example, U.S. Pat. Nos. 6,355,270; 6,140,127; and 6,053,171. The resulting amine, hydroxyl, or carboxy functional group can be used to attach a substrate to the surface.

The incubating step involves placing the enzyme composition and the substrate composition together under conditions wherein the enzyme is biologically active, to permit the enzyme to modify the substrate. For a sample that comprises one or more whole cells, the incubating may involve adding the substrate to the culture media of the cell, for example. For an sample that is a cell lysate, the incubating may involve mixing the enzyme and the substrate together. Factors required for enzymatic activity, such as a particular temperature or pH, salt concentration, co-factors such as Mg⁺², ATP, GTP, and the like, will generally be known for enzymes, and even when unknown, would be expected to be similar to the physiological microenvironment where the enzyme is active in vivo.

The reaction mixture is brought to a temperature sufficient to allow the enzymatic reaction to occur. This temperature can be between 0° C. and 100° C., more preferably, 0-75° C. or 0-50° C. for most organisms. In certain cases, especially enzymes from warm-blooded animals or humans, the temperature is in the range of about 35° C. and 40° C. In some cases, the temperature is physiological temperature, or about 37° C. Other temperatures contemplated include about 20, 21, 22, 23, 24, 25, 26, 27, 28, 29, 30, 31, 32, 33, 34, 35, 36, 37, 38, 39, 40, 41, 42, 43, 44, and 45° C. The pH of the reaction mixture is also adjusted to a pH sufficient to allow the enzymatic reaction to occur. The pH may be in the range of about 0 to 14, and more preferably, about 5 to about 9, or about 6 to about 8. In some cases, the pH is about 7.4.

The reaction mixture is allowed to react for at least a time sufficient to produce enough reaction product to be measured by the analytical machines. In some variations, aliquots are collected at different time points to assess the rate of the reaction, while in others, only one aliquot at one time point is collected. The length of time that the enzymatic reaction occurs will be dependent upon the enzyme of interest, its concentration and activity in the sample, and in the purposes of the measurements, and will be easily determined by the person of skill in the art, in view of this disclosure.

In general, reagents are added included in a sample and/or substrate to prevent enzyme or substrate degradation (e.g., protease inhibitors); preserve enzymatic activity (e.g, buffers, temperature, co-factors, salt concentration, ionic strength, pH, energy sources, and co-reagents); and prevent degradation of enzymatic reaction product (e.g., phosphatase inhibitors to prevent degradation of reaction products of kinases). With respect to preservation of enzymatic activity, prior literature that reports studies of enzymatic activity provides a rich source for information about buffers, pH, temperature, and other reaction conditions that are suitable for the same or similar enzymes for practicing methods of this invention. More generally, conditions that mimic an enzyme's natural environment (e.g., physiological temperature, pH, and ionic strength for many human or animal enzymes) are suitable for the present invention. Nonlimiting examples of reagents, buffers, salts, cofactors, inhibitors, include adenosine triphosphate (ATP), magnesium chloride, sodium chloride, phosphate buffers, iron, protease inhibitors, phosphatase inhibitors, Tris-HCl, HEPES, and chelating agents.

Exemplary protease inhibitors include, but are not limited to Na-p-tosyl-L-lysine chlormethyl ketone hydrochloride (TLCK), phenylmethylsulphonylfluoride (PMSF), leupeptin, pepstatin A, aprotinin, 4-(2-aminoethyl)benzenesulfonylfluoride hydrochloride (AEBSF), 6-aminohexanoic acid, antipain hydrochloride {[(S)-1-carboxy-2-phenylethyl]-carbamoyl-L-arginyl-L-valyl-arginal-phenylalanine}, benzamidine hydrochloride hydrate, bestatin hydrochloride, chymostatin, epoxysuccinyl-L-leucyl-amido-(4-guanidino)butane, ethylenediamine tetraacetic acid disodium salt, N-ethylmaleimide, and Kunitz trypsin inhibitor.

Exemplary phosphatase inhibitors include, but are not limited to, sodium fluoride, sodium orthovanadate, ocadaic acid, Vphen, microcystin, b-glycerophosphate, lacineurin, cantharidic acid, cyclosporin A, delamethrin, dephostatin, endothall, fenvalerate, fostriecin, phenylarsine oxide, and resmethrin.

Aliquots may be collected over a period time or one aliquot may be collected for a single analysis for a sample. The number of product molecules produced in an enzymatic reaction is dependent upon the incubation time. Therefore, the concentration or amount of product formed by the enzyme of interest may be normalized to the incubation time, which would allow for comparisons between data sets, time points, or samples. In some cases, the units of measurement for amount of product formed for an enzyme of interest are amount of product formed per unit time normalized to enzyme or lysate amounts (e.g., mol/s/K g or pmol/min/mg).

As applied to the disclosed methods, the term “quantitative” refers to the method's ability to provide an absolute measurement of enzymatic activity that can be compared to measurements taken at a different time or place. Quantitative measurements are more valuable for many purposes than relative measurements that can only be compared to other measurements taken at the same time that may yield information such as a ratio. In some embodiments, the use of a measured quantity of an internal standard permits quantitative calculation of the activity of an enzyme in a sample.

In some cases, the incubating further includes inhibitors added prior to or contemporaneous to starting the enzymatic reaction. The inhibitors can be specific inhibitors for one or more enzymes in the sample, other than the enzyme being measured. In some cases, the inhibitor is a protease inhibitor. Protease inhibitors serve to inhibit degradation of the enzyme or degradation of protein substrates and products. More generally, in some variations of the invention, the method includes the addition of factors that are necessary for the enzymatic reaction, or that improve the enzymatic reaction, or that prevent degradation of the product.

The term “isolating” refers to separating the particle from the enzymatic reaction mixture and, more importantly (in the case of polypeptide substrates), substantially all proteins. This isolation can be by any means, such as filtration, centrifugation, magnetic separation, or the like. The particles can be partially isolated by these physical means, such as filtering, centrifuging, or separating using a magnetic field, then washed with a solvent or buffer solution to remove any remaining proteins.

The product and unreacted substrate are then contacted with a cleavage agent to produce fragments of the product and any unreacted substrate. The term “cleavage agent” refers to a reactant that can degrade the substrate and/or product into fragments. The cleavage agent can be a protein, such as a protease, lypase, or glycosylase or can be a chemical agent. Non-limiting examples of proteases include trypsin, Asp-N, chymotrypsin, Lys-C, Lys-N, Arg-C, glutamyl endopeptidase, proline endopeptidase, proteinase K, thrombin, pepsin, and combinations thereof. Non-limiting examples of chemical agents include 2-(2-nitrophenylsulfenyl)-3-methylindole, cyanogen bromide, formic acid, hydroxylamine, iodosobenzoic acid, 2-nitro-5-thiocyanobenzoic acid, and combinations thereof. In some cases, the cleavage agent can simultaneously cleave the product and substrate into fragments and cleave the product, or resulting fragments, from the surface of the particle. In other cases, the product and unreacted substrate are cleaved from the surface of the particle prior to treatment with the cleavage agent. The means by which the product and unreacted substrate can be cleaved from the surface of the particle depends upon how the product and/or substrate is attached to the surface. Non-limiting examples of conditions for detaching a product and/or unreacted substrate from the surface of the particle includes acidic or basic hydrolysis, oxidation, and reduction.

The fragments produced from the contacting of the cleavage agent are suitable analytes for analysis by mass spectrometry. The fragments can optionally be purified prior to analysis, using any suitable purification means, including chromatography. The fragments can be of any molecular weight, but, in preferred embodiments, will typically have a mass of less than about 5 kDa. The molecular weight of the fragments can be less than about 4 kDa, less than about 3 kDa, less than about 2 kDa, less than about 1 kDa, or less than about 500 Da.

In some cases, the methods further include purifying the fragments before the analyzing step to provide a purified set of fragments for analysis. Any techniques that are useful for chemical or biochemical separation may be used for the purifying step, including the use of chromatographic techniques, affinity purification materials and methods, electrophoresis techniques, and the like. In certain cases, the purification is done by high pressure liquid chromatography (HPLC). In some cases, chromatography specifically designed for the purification or enrichment of phosphopeptides is used. Such techniques include immobilized metal affinity chromatography (IMAC), TiO₂ and ZrO₂ chromatography. In some cases, the separation is performed using magnetic beads

In various cases, an internal standard can be added to the fragments prior to analyzing by MS. In variations where an internal standard is used, the quantity of the first product of the enzymatic reaction can be calculated by comparing mass spectrometric measurements of the first product and the internal standard. Alternatively, the internal standard can be added prior to cleaving the product and unreacted substrate from the particle. In specific cases, the internal standard is added during the incubating.

Internal standards include, but are not limited to, isotopically labeled peptides, and compound structurally related to the product or substrate to be quantified. In some cases, only one internal standard is added; in other cases, two or more internal standards are added.

Isotopically labeled peptides are peptides that incorporate at least one rare isotope atom, such as a ¹³C, ¹⁵N, and/or ²H atom, so as to give the labeled peptide an essentially identical molecular structure but different molecular weight than a fragment of the substrate or product. Stable isotopes (non-decaying isotopes or isotopes with very long half lives) are preferred, and among isotopes that do decay, those that decay to give off lower level radiation are preferred. Incorporation of one or more isotopes can be accomplished in a variety of ways. Amino acids containing one or more ¹³C, ¹⁵N and/or ²H can be obtained from commercial sources such as Sigma-Aldrich (Milwaukee, Wis., USA) and, using a peptide synthesizer, these isotopically labeled amino acids can be integrated into a peptide sequence. Isotopically labeled peptides can be produced by recombinant DNA technology. Organisms such as bacteria are transfected with a plasmid bearing a sequence for a peptide that may be an internal standard. By growing bacteria in media in which one amino acid is replaced by its isotopically labeled counterpart, it is possible to obtain the labeled peptide using standard purification methods. Such methods are described in U.S. Pat. No. 5,885,795 and U.S. Pat. No. 5,151,267, each of which is incorporated by reference in its entirety.

The analysis of the fragments can occur by tandem mass spectrometry, which involves a first mass spectrometry analysis to isolate a fraction of the ionized sample that contains the product; fragmenting the product in the fraction; and performing a second mass spectrometry analysis after the fragmenting to measure at least one fragmented fragment from the product, wherein the measurement indicate the quantity of the product y. The analysis may also be performed by conventional mass spectrometry, in which matrix assisted laser desorption ionization (MALDI) or electrospray ionization is coupled with single mass analyzers such as time of flight (TOF), quadrupoles, sectors, or ion traps. In some variations, the measurement is performed by quantitative evaluation of the unfragmented molecular ions.

MS analysis involves the measurement of ionized analytes in a gas phase using an ion source that ionizes the aliquot, a mass analyzer that measures the mass-to-charge (m/z) ratio of the ionized aliquots, and a detector that registers the number of ions at each m/z value. The MS apparatus may be coupled to separation apparatus (e.g., such as chromatography columns, on-chip separation systems, and the like) to improve the ability to analyze complex mixtures.

Tandem MS (interchangeably called MS/MS herein) analysis involves a gas phase ion spectrometer that is capable of performing two successive stages m/z-based discrimination of ions in an ion mixture. This includes spectrometers having two mass analyzers as well as those having a single mass analyzer that are capable of selective acquisition or retention of ions prior to mass analysis. These include ion trap mass spectrometers, ion trap-TOF mass spectrometers, TOF-TOF mass spectrometers, triple quadrupoles, quadrupole-TOF (Q-TOF), Fourier transform ion cyclotron resonance mass spectrometers, orbitrap mass spectrometers, and combinations thereof.

A range of ions with different mass-to-charge (m/z) values can be trapped simultaneously in a quadrupole ion trap by the application of a radio frequency (RF) voltage to the ring electrode of the device. The trapped ions all oscillate at frequencies that are dependent on their m/z, and these frequencies can be readily calculated. Tandem MS is then performed by carrying out three steps. First, the analyte ions having the single m/z of interest (parent ions) are isolated by changing the RF voltage applied to the ring electrode and by applying waveforms (i.e. appropriate ac voltages to the endcap electrodes) with the appropriate frequencies that resonantly eject all the ions but the m/z of interest. Second, the isolated parent ions are then resonantly excited via the application of another waveform that corresponds to the oscillation frequency of the parent ions. In this way, the parent ions' kinetic energies are increased, and they undergo energetic collisions with the background gas (usually helium), which ultimately result in their dissociation into product ions. Third, these product ions are then detected with the usual mass analysis techniques in MS.

Multiplexed MS/MS refers to measuring the activity of several enzymes within the same assay. Multiple reaction monitoring (MRM) may be used for multiplexed MS/MS analysis, wherein MRM is performing several MS/MS measurements simultaneously on ions of multiple m/z ratios.

In some variations, collision induced dissociation (CID) may be employed during MS analysis. CID is a mechanism by which to fragment molecular ions in the gas phase. The molecular ions are usually accelerated by some electrical potential to high kinetic energy in the vacuum of a mass spectrometer and then allowed to collide with neutral gas molecules (often helium, nitrogen or argon). In the collision some of the kinetic energy is converted into internal energy which results in bond breakage and the fragmentation of the molecular ion into smaller fragments. These fragment ions can then be analyzed by a mass spectrometer. CID and the fragment ions produced by CID are used for several purposes. By looking for a unique fragment ion, it is possible to detect a given molecule in the presence of other molecules of the same nominal molecular mass, essentially reducing the background and increasing the limit of detection.

When the activity of more than one enzyme is measured, a mass spectrometer can be set up so that it analyzes individually each set of fragments. This can be accomplished using tandem MS analysis, wherein the sample is may be fractioned into a specific mass range, correlating with the substrate and/or product of a first enzyme, and separated from the rest of the sample, and then the specified molecules are broken into fragments and analyzed for amount of product formed by the first enzyme. A fraction having a different mass range can then be isolated from the same sample with the second mass range, correlating with a second enzyme's substrate and/or product, and analyzed. The means of doing multiple analyses of analytes by tandem mass spectrometry are described, for example, in U.S. Pat. Nos. 5,206,508; 6,649,351; 6,674,096; and 6,924,478, each of which is incorporated in its entirety by reference.

The MS analysis results in a spectrum of ion peaks with relative intensities relating to their concentration in the aliquot. When an internal standard of known quantity or concentration and volume is added to the sample, the relative signal strengths of the peptide internal standard peak and product peak may be calculated to give an enzyme activity in relative terms. Multiplication of the ratio of signal strengths between the internal standard and peptide product with the known concentration of the standard yields a quantitative measurement of the product, which in turn represents a quantitative measurement of the activity of the enzyme. For example, if the ratio of peptide product to internal standard is 1:0.5, the concentration of the peptide product will be two times the concentration of the internal standard. In variations where more than one enzyme is being evaluated, each enzyme's activity can be assessed by the same means of measuring the ratio of a fragment associated with the first enzyme's product to an internal standard and independently, the ratio a fragment associated with the second enzyme's product to the same or a different internal standard.

Since the enzyme activity can be given in absolute terms, the enzymatic activity of particular enzymes can be compared from sample to sample, allowing for the assessment of enzymatic activity from one sample, or patient, to another; or from one treatment to another. This may allow for the rapid diagnosis of a particular diseases state or for the assessment of the efficacy of a particular treatment in view of a different treatment.

Different enzymatic activities can be analysed in the same mass spectrometric analysis because each enzyme produces a product with a unique mass and charge (e.g., a phosphopeptide), which upon fragmentation produces fragment ions of distinct mass. A mass spectrometer can distinguish different products based on the mass of the enzymatic product, and a tandem mass spectrometer can sequence the product (e.g., the phosphopeptide) to unambiguously identify and quantify different enzymatic activities simultaneously.

The methods described herein may be used to assess or screen an organism, human, or animal subject for abnormalities by detecting aberrant enzyme activity. By understanding the connection between specific enzymes and disease states, the methods allow for rapid determination of one or more enzyme activities which may be correlated to specific disease states. In some cases, more than one aberrant enzyme may be detected. By collecting samples from an organism or subject of interest and applying that sample to the methods disclosed herein one may be able to diagnose or screen for abnormalities which may then be linked to specific disease states. The aberrant enzyme activity may be detected by comparing the enzyme activity of the sample from the organism with a reference sample. Reference samples may be from the same organism at a different time or from a different location in the organism, or may be from a different organism of the same species, or a statistical measurement calculated from measurements of samples of cells of the same cell type, from multiple organisms of the same species, to provide an average for that organism and that cell type.

The detection and effective therapeutic modulation (stimulation, up-regulation, inhibition, or blockade) of signal transduction pathways in human diseases, including, but not limited to, cancer, diabetes, allergies, inflammation, and neurodegenerative diseases, is seriously hampered by inadequate tools to quantify changes in pathway activation status. The techniques described here, in one embodiment, enable the measurement of signal transduction pathway activity in a biological sample (such as a tissue, fluid, or cell sample) with the sensitivity, specificity, and precision needed for providing clinically useful information. This analytical strategy may be applied to any protein or enzyme whose product or substrate is amenable to mass spectrometric detection. In preferred variations, at lease one selective substrate of the target enzyme is available. Enzymes and substrates/products involved in a signal transduction pathway provide clinically useful information about the pathway. Because this method is based upon a biochemical (e.g., enzymatic) reaction that amplifies the signal of the target molecule, it could be described as a proteomic analytical equivalent the polymerase chain reaction (PCR) used to amplify nucleic acid sequences.

The mechanism of action of many pharmaceutical agents (as well as lead, pre-clinical, and clinical candidate compounds) is to modulate enzymatic activity, which is a major factor in controlling cellular and tissue biochemistry. By providing a rapid, sensitive, specific, and optionally multiplex means for analyzing enzyme activities involved in signal transduction, metabolism, and related biochemical processes, the materials and methods of the invention are useful for both drug research and development and drug prescription, administration, and patient monitoring. For example, in the field of drug development, the materials and methods of the invention are useful for assessing the biological activity of a compound on a target pathway, and also for assessing the biological activity of the compound on non-target pathways that may be relevant to drug metabolism/clearance, drug toxicity, drug-drug interactions, and side-effects.

In a typical drug screening, the activity of a system is independently measured in the absence and presence of a test compound. The affect of that test compound is evaluated as a comparison between the measured activity in the absence of the compound and the activity in the presence of the compound. The methods disclosed herein are a means of measuring the effect of a potential drug candidate in a biological system by providing quantitative measurements of activities of one or more enzymes of interest in a biological system.

It is well established that not all patients that have been diagnosed with a disease or condition will respond to the same medication in the same way, or at the same dose, or with the same side effects. The materials and methods of the invention have utility in this clinical setting as well, e.g., to identify the subpopulation of patients that are more likely to benefit from using a particular drug, targeting a specific pathway, selecting a dose or dosing regimen, and minimizing unnecessary side effects. In these ways, the materials and methods of the invention are useful for improving personalized disease therapy. Appropriateness of a particular drug may be predicted by analyzing a biological sample from a patient to determine the activity of the protein(s) on which the target enzyme acts.

Specific aberrant enzyme activity has been associated with many disease states. Enzyme activity which is aberrant is activity that is either higher or lower than an enzyme's usual activity in a population (or samples from a population) not affected by a particular disease state. By being able to quantitatively measure enzyme activity in a manner that allows meaningful comparisons between sample sets, it may be possible to identify a particular disease state, select a more effective therapy, measure efficacy of treatments for diseases, and compare different treatments. The ability to measure enzymatic or protein activity with exquisite sensitivity also has indications for predicting the future occurrence of, or early diagnosis of, diseases at a time before other, more noticeable signs or symptoms of the disease present themselves, permitting earlier treatment, prophylaxis, and/or lifestyle management decisions to prevent or delay the onset of disease. For example, cancer, diabetes, allergic reactions, inflammation, neurodegenerative diseases, metabolic disorders, senescence, and many other disease states are known to be related to aberrant enzymatic activity.

Therefore, in some embodiments, the methods described herein are directed toward characterizing a disease, disorder, or abnormality. A particular disease state may not exhibit itself the same way in all subjects. Therefore, a measurement of the activity of the enzyme or enzymes implicated in a particular disease may yield useful information with respect to the manner in which a particular disease is manifested in a specific subject. The activity of the enzyme or enzymes of the subject is then compared to the activity of a reference measurement. In some cases, the comparison is made over time, and can be used to assess the efficacy of a particular therapy or to evaluate the progression of a particular disease. In certain specific embodiments, the comparison is used to select an appropriate composition or compound for administration to the subject which is specific for the particular aberrant activity measured using the methods disclosed herein. In subjects where the aberrant activity is measured in certain enzymes, one compound or composition will be most effective, while other subjects with different aberrant activity will be best treated by a different set of compositions or compounds. The materials and methods of the invention provide information and guidance for selection of more effective compositions or compounds.

Additional aspects and details of the disclosure will be apparent from the following examples, which are intended to be illustrative rather than limiting.

### EXAMPLES

### Example 1

All examples described in International Patent Publication No. WO 07/127,767 are incorporated herein in their entirety as Example 1. The WO 07/127,767 application is incorporated by reference in its entirety.

### Example 2

4EBP1 was used as the substrate for kinase reactions. 4EBP1 is a physiological substrate of mTORC1, a Ser/Thr protein kinase implicated in cell growth and metabolism and a drug target for cancer treatment (Wullschleger et al., Cell 124:471-84 (2006)). Rat GST-4EBP1 fusion protein was immobilized to glutathione SEPHAROSE® beads by incubating recombinant GST-4EBP1 with the beads for two hours at 4° C. Human proteins were synthesised with N-terminal GST and His tags separated by a PreScission protease cleavage site. Purification was performed with glutathione S-sepharose chromatography. Protease cleavage resulted in soluble His-Tagged proteins, which were then linked to TALON® magnetic beads (Invitrogen), following the manufacturer's protocol. These immobilized proteins were used as substrates for kinase reactions using total cell lysates as an enzyme source.

NIH-3T3 cells were grown in DMEM medium supplemented with 10% fetal calf serum, and lysed in lysis buffer (1% v/v Triton X-100, 50 mM Tris.HCl pH 7.4, 150 mM NaCl, 1 mM EDTA, 10 μM leupeptin, 0.5 mM okadaic acid, 0.5 mM NaF, 1 mM Na₃VO4, 10 μg/ml TLCK, 1 mM DTT, 1 μg/ml pepstatin, 0.05 TIU/mg aprotinin, 17 μg/ml PMSF). Lysates were centrifuged at 12,000×g for 10 minutes at 4° C.

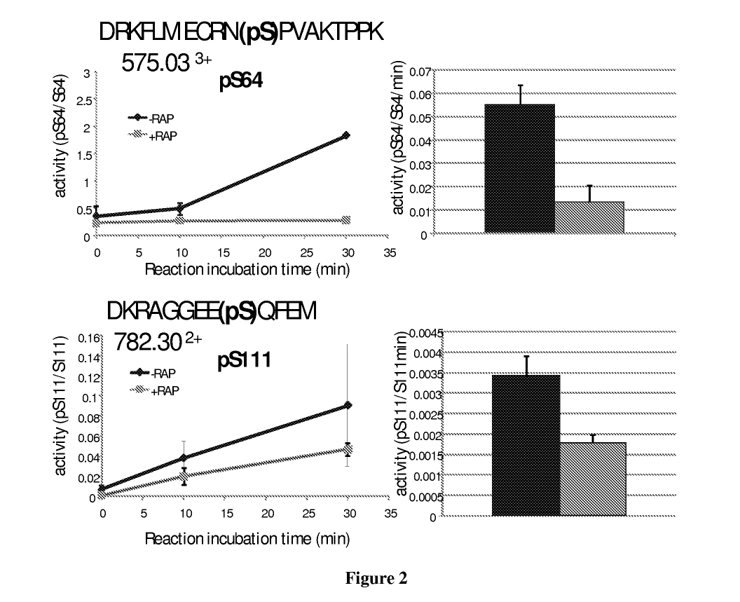

Kinase reactions were carried out in 50 μl reaction volumes containing different amounts of protein (as measured by the Bradford assay) and beads containing immobilized substrate (e.g., 4EBP1) in reaction vessels that also contained Tris.HCl pH 7.4, 50 mM NaCl, 100 μM ATP and 5 mM MgCl₂. Reactions were performed for the times indicated in FIG. 2 and stopped with 0.1% formic acid. Beads containing reaction products were collected by magnetic separation using a magnet, and the solvent was discarded. The beads were then washed three times with 50 mM ammonium bicarbonate pH 8.8 and submitted to proteolytic digestion using Asp-N with overnight incubation.

Two phosphopeptides derived from Asp-N proteolytic cleavage of this protein were readily detectable by LC-MS/MS after incubation of immobilised 4EBP1 with cell lysates and ATP/Mg²⁺. The ion intensities of these phosphopeptides (SEQ ID NOs: 1 and 2), normalised to the intensities of their non-phosphorylated counterparts, increased with prolonged incubation time (FIG. 2). The residues phosphorylated in these phosphopeptides corresponded to S64 and S111 of the rat 4EBP1 sequence. S64 in 4EBP1 is a known substrate for the kinase activity of mTORC1, and accordingly, treatment of cells with rapamycin, an mTORC1-specific inhibitor, resulted in a complete abolishment of kinase activity towards S64 (SEQ ID NO: 1) in total cell lysates, whereas kinase activity towards S111 (SEQ ID NO: 2) was only partially inhibited by rapamycin. These results indicate that kinase activity towards 4EBP1 S64 is rapamycin sensitive. This activity could be mediated by mTORC1 or by other protein kinases downstream mTORC1, but regardless of its molecular nature, it constitutes a readout of rapamycin target inhibition, most probably mTORC1 activity.

### Example 3

Mass spectrometry was used to multiplex the measurement of protein kinase activities, using BAD as a substrate for in vitro reactions. BAD is a known substrate of the protein kinase AKT/PKB, which is downstream of PI3K and upstream mTORC1. Mouse His-Tag-BAD protein was immobilized to SEPHAROSE® beads. In subsequent experiments, human proteins were synthesised with N-terminal GST and His tags separated by a PreScission protease cleavage site. Purification was performed with glutathione S-sepharose chromatography. Protease cleavage resulted in soluble His-Tagged proteins, which were then linked to TALON® magnetic beads (Invitrogen), following the manufacturer's protocol. These immobilized proteins were used as substrates for kinase reactions using total cell lysates as an enzyme source.

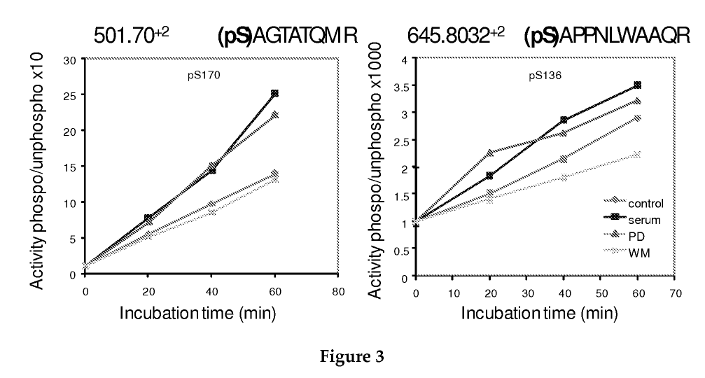

Starved NIH-3T3 cells, pre-treated or not with the inhibitors WM or PD, were stimulated with 10% fetal calf serum for 5 minutes prior to cell lysis. Cell lysates were mixed with immobilized BAD and ATP/Mg²⁺ and reactions allowed to occur at 37° C. for the times indicated in FIG. 3. Reactions were stopped, and the beads washed three times to remove lysate components and to buffer exchange to 50 mM ammonium bicarbonate. For each wash, beads were isolated from the reaction by magnetic separation using a magnet. Immobilised phosphorylated BAD was digested with trypsin and resultant peptides analysed by LC-MS and LC-MS/MS. The indicated phosphorylated peptides containing sites of phosphorylation pS170 (SEQ ID NO: 3) and pS136 (SEQ ID NO: 4) of the mouse BAD sequence showed increased signals as a function of reaction time. These observed activities increased when serum was added to the medium prior to lysis and were sensitive to the addition of wortmannin (WM) but not the MEK inhibitor PD98059 (PD), thus arguing that these activities are a measure of WM target inhibition, probably PI3K signalling activity, a known target of WM, and are probably downstream PI3K. In contrast, these activities were not sensitive to PD, indicating that the BAD kinase activities measured by LC-MS were downstream the PI3K/Akt pathway but not the MEK/Erk pathway.

### Example 4

In occasions when it is desirable to quantify absolute activities, internal standards (ISs) can be used. These ISs are isotopically labeled with non-radioactive heavy isotopes of carbon, nitrogen or hydrogen and have the same sequence as the product and substrate (e.g., phosphopteptide and peptide, when the enzyme is a kinase) to be quantified. This type of analysis involves mixing immobilized substrates with a biological sample taken from a cell lysate or tissue homogenate together with ATP, Mg²⁺ and other reagents and buffers as needed to perform the enzymatic reaction. After allowing the reaction to occur for 10 to 30 minutes, the products of the reaction are separated from the sample by magnetic separation or by other means such as centrifugation. The beads containing the reaction product are then washed with a buffer of neutral pH which is compatible with mass spectrometry, such as ammonium bicarbonate. The appropriate IS (e.g., having the same sequence as the product) is then added at a known concentration and volume to the vessel containing the reaction products. This mixture is then digested with a protease, such as Asp-N or trypsin. The fragments of the product of the reaction, the unreacted substrate, and the IS is then analysed by LC-MS or LC-MS/MS. The intensities of these analytes are recorded. Suitable intensity readouts are area under the curve or heights of peaks produced by the analyte in LC-MS or LC-MS/MS runs. Quantification of product of the enzymatic reaction is derived by dividing the intensity value of the product by the intensity of its IS multiplied by the amount of IS added to the vessel. The amount of product to the amount of remaining substrate can also be normalized in a similar fashion. This allows for normalization of differences in enzyme activities. In these cases, the amount of unreacted substrate is quantified by dividing the intensity value of the unreacted substrate by the intensity of its IS multiplied by the amount of IS added to the vessel. A normalized activity value is given by the amount of product divided by the amount of unreacted substrate.

### Example 5

P31-Fuj AML cells were seeded at a density of 500000 cells/mL. The following day, cells were either treated with 10 mM LY204002, a PI3K inhibitor, or equivalent concentration of DMSO (as control) for 1 hr prior to harvesting. Cells were washed twice in ice cold PBS containing 100 mM NaVO₃ and 1 mM NaF and were subsequently harvested and lysed in the following lysis buffer: 40 mM Hepes pH 7.5, 120 mM NaCl, 1 mM EDTA, 10 mM pyrophosphate, 10 mM glycerophosphate, 50 mM NaF, 0.5 mM orthovanadate, 0.3% CHAPS, and complete protease inhibitors (Roche).

Kinase reactions contained 30 mg lysate material and were performed under standard conditions as described in a preceding example. The reaction was started by addition of 10 mL (5 mg) of TALON® beads having immobilized substrates on the surface, each bead having a single type of substrate immobilized on it. The reactions were performed in the presence of either ‘pool 1’—a mixture of beads, each having one of SMAD1, Myc, Annexin or Beta Catenin substrates immobilized on its surface—or ‘pool 2’—a mixture of beads, each having one of S6, Foxo3A, 4EBP1 or p53 substrates immobilized on its surface. After allowing the reactions to occur in the presence of ATP and Mg²⁺, samples were trypsin digested and subject to LC-MS analysis. The data in the following table shows the relative kinase activity of the phosphorylated peptides detected, each representing a kinase reaction. The reported kinase activities were normalized to the mean activities from control and treated samples and log 2 converted.

TABLE

| | | DMSO | |
|---|---|---|---|
| | Phophopeptide | (Control) | Ly294002 |
| Pool 1 | SMAD-1 | 0.449256 | −0.65594 |
| | (RVESPVLPPVLVPR | | |
| | SEQ ID NO: 5) | | |
| | SMAD-1 | 0.844462 | −2.2905 |
| | (HSEYNPQHSLLAQFR | | |
| | SEQ ID NO: 6) | | |
| | myc | −0.14067 | 0.128166 |
| | (CHVSTHQHNYAAPPSTR | | |
| | SEQ ID NO: 7) | | |
| | myc | −0.16639 | 0.149169 |
| | (FELLPTPPLSPSR | | |
| | SEQ ID NO: 8) | | |
| | myc | 0.065397 | −0.0685 |
| | (FELLPTPPLSPSRR | | |
| | SEQ ID NO: 9) | | |
| | myc | 0.279323 | -0.34671 |
| | (VKLDSVR | | |
| | SEQ ID NO: 10) | | |
| | Annexin | −0.61339 | 0.429044 |
| | (MYGISLCQAILDETKGDYEK | | |
| | SEQ ID NO: 11) | 0.681911 | −1.33739 |
| | Beta Catenin | 0.681911 | −1.33739 |
| | (AAVSHWQQQSYLDSGIHSGATTTAPSLSGK | | |
| | SEQ ID NO: 12) | | |
| | Beta Catenin | 0.414375 | −0.58364 |
| | (TSMGGTQQQFVEGVR | | |
| | SEQ ID NO: 13) | | |
| Pool 2 | S6 | 0.409016 | −0.57299 |
| | (DIPGLTDTTVPR | | |
| | SEQ ID NO: 14) | | |
| | S6 | −0.00773 | 0.007685 |
| | (DIPGLTDTTVPRR | | |
| | SEQ ID NO: 15) | | |
| | foxo | 0.447365 | −0.65188 |
| | (WPGSPTSR | | |
| | SEQ ID NO: 16) | | |
| | foxo | 0.164209 | −0.18533 |
| | (HNLSLHSR | | |
| | SEQ ID NO: 17) | | |
| | foxo | 0.448456 | −0.65422 |
| | (AVSMDNSNKYTK | | |
| | SEQ ID NO: 18) | | |
| | 4ebp1 | 0.963738 | −4.33226 |
| | (TPPRDLPTIPGVTSPSSDEPPMEASQSHLR | | |
| | SEQ ID NO: 19) | | |
| | p53 | 0.814518 | −2.05116 |
| | (SVTCTYSPALNK | | |
| | SEQ ID NO: 20) | | |
| | p53 | 0.987228 | −5.82605 |
| | (ALPNNTSSSPQPK | | |
| | SEQ ID NO: 21) | | |
| | p53 | 0.911958 | −3.07822 |
| | (CSDSDGLAPPQHLIR | | |
| | SEQ ID NO: 22) | | |
| | p53 | 0.737407 | −1.58717 |
| | (RALPNNTSSSPQPK | | |
| | SEQ ID NO: 23) | | |

### Example 6

### Comparison of Multiplexed Analysis on Immobilized Substrates and Peptide Analysis (as Disclosed in WO 07/127,767)

The specificity of protein kinases for their substrates is based on the amino acid sequence around the site being phosphorylated. ‘Long range’ protein-protein interactions between the kinase and the substrate confer additional specificity. Short peptides can be used as substrates for kinase assays but when several kinases are present in the assay, such as when total cell lysates are used as the enzyme source, kinases may contribute to non-specific background activity. The specificity of a kinase assay can be enhanced by using the natural substrates of the kinases to be assayed, as this allows for long range kinase-substrate interactions to occur.

Data from a peptide kinase assay using the Aktide peptide (a peptide substrate with sequence RPRAATF—SEQ ID NO: 24—known to provide a readout of PI3K pathway activation) was compared with the data from a multiplex kinase assay using full length protein substrates. For this, P31-Fuji cells (an acute myeloid leukaemia cell line) were lysed and the kinase activity in total cell lysates measured using the protocols disclosed in WO 07/127,767 or using the protocols disclosed herein. Before lysis, cells were divided into two groups—(1) control and (2) treated with LY294002 (an inhibitor of PI3K which controls downstream protein kinases that phosphorylate the Aktide).

In the peptide assay, LY treatment resulted in about 85% reduction on the kinase activity towards the Aktide. In contrast, in the assay with full length proteins, the kinase activities towards Ser94 (on the substrate termed 4EBP1) and on Ser121 (on the substrate termed p53) were reduced substantially more, by 95% and 99%, respectively. These results indicate that about 10% to 15% of the activity towards the Aktide peptide is not downstream PI3K, given that this residual activity is not inhibited by LY294002. These data also indicate that full length protein substrates provide additional specificity for kinase assays, and that kinase activities on 4EBP1 and p53 amino acid residues are good readouts of PI3K activation.

1. A method of measuring the activity of a first enzyme in a sample that contains one or more enzymes, the method comprising:
a) incubating the sample with a first particle in a mixture to start a first enzymatic reaction,
wherein the first particle has, attached to its surface, a first substrate for a first enzyme that is known or suspected of being present in the sample,
wherein the first enzyme is a kinase, and
wherein the incubating is under conditions effective to permit a first enzymatic reaction involving the first enzyme and the first substrate to produce a first product, the first product being attached to the surface of the first particle;
b) isolating the first particle from the mixture of (a), wherein the isolated first particle includes, attached to its surface, first product and unreacted first substrate;
c) contacting the first product and the unreacted first substrate with a cleavage agent under conditions sufficient to cleave the first product and the unreacted first substrate into a first set of fragments; and
d) analyzing the first set of fragments of (c) by mass spectrometry to determine the quantity of the first product that was produced in (a),
wherein the quantity of the first product provides a measurement of the activity of the first enzyme in the sample.

2. The method of claim 1, wherein the sample contains a plurality of enzymes.

3. The method of claim 1, wherein the contacting with the cleavage agent simultaneously detaches the first product and the unreacted first substrate from the surface of the first particle.

4. The method of claim 1, wherein the first product and the unreacted first substrate are detached from the surface of the first particle prior to contacting with the cleavage agent.

5. The method of claim 1, further comprising adding an internal standard of known molecular weight to the first set of fragments prior to analyzing.

6. The method of claim 5, wherein the internal standard is added prior to the contacting step.

7-8. (canceled)

9. The method of claim 1, wherein the final substrate has a molecular weight of at least 25 kDa.

10. (canceled)

11. The method of claim 1, wherein the first enzyme enzymatically reacts with a protein, and wherein the first substrate comprises a polypeptide that includes the complete amino acid sequence of the protein.

12. The method of claim 1, wherein the first enzyme enzymatically reacts with a protein that includes a plurality of domains, and wherein the first substrate comprises a polypeptide that includes the complete amino acid sequence of a domain with which the enzyme reacts.

13-14. (canceled)

15. The method of claim 1, wherein the first enzymatic reaction is performed in a mixture that includes the first particle and the sample, and wherein the first particle has a size or density that causes the particle to remain in suspension in the mixture during the first enzymatic reaction.

16. (canceled)

17. The method of claim 15, wherein the first particle has a diameter of 2 μm or less.

18. (canceled)

19. The method of claim 15, wherein the first particle is magnetic, and the isolating comprises magnetic separation.

20-22. (canceled)

23. The method of claim 1, wherein the first enzyme is a protein kinase, and the first substrate comprises a polypeptide.

24-26. (canceled)

27. The method of claim 23, further comprising purifying the first set of fragments prior to the analyzing step.

28-29. (canceled)

30. The method of claim 1, wherein the first substrate comprises at least one amino acid, and is attached to the first particle via a side chain of an amino acid of the first substrate.

31-34. (canceled)

35. The method of claim 23, wherein the cleavage agent comprises at least one protease.

36-40. (canceled)

41. The method of claim 1, wherein the sample comprises a cell lysate that comprises enzymes from a cell.

42. (canceled)

43. The method of claim 41, wherein the sample comprises a lysate from 1000 or fewer cells.

44-45. (canceled)

46. The method of claim 1, wherein the sample is from a human or animal subject suspected of having a disease characterized by changes in the activity of an enzyme involved in a cellular process, and wherein the enzyme involved in the cellular process is the first enzyme.

47. The method of any one of claim 46, wherein the sample or the enzymatic reaction further comprises one or more enzyme inhibitors to inhibit one or more enzymes, other than the first enzyme, that may be present in the sample.

48. The method of claim 47, wherein the one or more enzyme inhibitors comprise a protease inhibitor.

49. The method of claim 47, wherein one or more enzyme inhibitors comprise a phosphatase inhibitor.

50. The method of claim 1, wherein the analyzing comprises quantitatively determining the activity of the first enzyme.

51. The method of claim 50, wherein the determining of the quantitative activity of the first enzyme comprises comparing the quantity of the first product to the quantity of the unreacted first substrate.

52. The method of claim 50, wherein the determining of the quantitative activity of the first enzyme comprises comparing the quantity of the first product to the quantity of an internal standard.

53. The method of claim 1, further comprising measuring the activity of a second enzyme known or suspected of being in the sample,
wherein the first particle further comprises a second substrate attached to its surface;
wherein the first particle and sample are incubated under conditions effective to permit a second enzymatic reaction involving the second enzyme and the second substrate to produce a second product, the second product being attached to the first particle;
wherein the isolated first particle further includes, attached to its surface, the second product and unreacted second substrate;
wherein the second product and unreacted second substrate are contacted with a cleavage agent under conditions sufficient to cleave the second product and unreacted second substrate into a second set of fragments; and
wherein the second set of fragments are analyzed by mass spectrometry to determine the quantity of the second product that was produced, wherein the quantity of the second product provides a measurement of the activity of the second enzyme in the sample.

54. The method of claim 53, wherein the second substrate is specific for the second enzyme.

55. The method of claim 1, further comprising detecting a second activity of the first enzyme,
wherein the first particle further comprises a second substrate attached to its surface;
wherein the first particle and sample are incubated under conditions effective to permit a second enzymatic reaction involving the first enzyme and the second substrate to produce a second product, the second product being attached to the first particle;
where the isolated first particle further includes, attached to its surface, the second product and unreacted second substrate;
wherein the second product and unreacted second substrate are contacted with a cleavage agent under conditions sufficient to cleave the second product and unreacted second substrate into a second set of fragments; and
wherein the second set of fragments are analyzed by mass spectrometry to determine the quantity of the second product that was produced, wherein the quantity of the second product provides a measurement of the activity of the first enzyme in the sample towards the second substrate.

56. The method of claim 53, wherein the incubating of the first and second substrate with the sample occurs simultaneously.

57. The method of claim 53, wherein the conditions effective to permit the first reaction are different from the conditions effective to permit the second reaction, and wherein the first particle is incubated with the sample sequentially under at least two reaction conditions to permit the first and second enzymatic reactions.

58-59. (canceled)

60. The method of claim 53, wherein the analyzing comprises quantitatively determining the activity of the second enzyme toward the second substrate.

61-62. (canceled)

63. The method of claim 1, further comprising measuring the activity of a second enzyme known or suspected of being in the sample, comprising:
incubating the sample with a second particle in an incubating mixture to start a second enzymatic reaction, wherein the second particle has, attached to its surface, a second substrate for the second enzyme, and wherein the incubating is under conditions effective to permit a second reaction involving the second enzyme and the second substrate to produce a second product, and the second product and unreacted second substrate are attached to the surface of the second particle;
isolating the second particle from the incubating mixture;
contacting the second product and unreacted second substrate with a cleavage agent under conditions sufficient to cleave the second product and unreacted second substrate into a second set of fragments; and
analyzing the second set of fragments by mass spectrometry to determine the quantity of the second product produced, such that the quantity of the second product provides a measurement of the activity of the second enzyme in the sample.

64. The method of claim 63, wherein the first and second enzymatic reactions occur under the same conditions, and wherein the incubating of the first and second particles with the sample occurs simultaneously.

65. The method of claim 63, wherein the conditions effective to permit the first reaction are different from the conditions effective to permit the second reaction, and wherein the incubating occurs sequentially under at least two reaction conditions, to permit the first and second enzymatic reactions.

66-70. (canceled)

71. A method of determining the activity of a first enzyme and a second enzyme in a sample that contains two or more enzymes, comprising:
a) incubating the sample with a first particle in an incubating mixture to start a first enzymatic reaction, wherein the first particle has, attached to its surface, a first substrate having a first domain that is specific for the first enzyme and a second domain that is specific for the second enzyme, and the incubating is under conditions effective to permit a first enzymatic reaction involving the first enzyme and the first domain to produce a first product domain,
b) incubating the sample with the first particle in an incubating mixture under conditions effective to permit a second enzymatic reaction involving the second enzyme and the second domain to produce a second product domain, such that the first particle includes, attached to its surface, a product mixture comprising unreacted first substrate and first substrate that includes a first product domain and/or a second product domain;
c) isolating the first particle from the incubating mixture;
d) contacting the first particle with a cleavage agent under conditions to cleave the product mixture into a first set of fragments; and
e) analyzing the first set of fragments of (d) by mass spectrometry to determine the quantity of first product domain and second product domain,
wherein the quantity of the first product domain provides a measurement of the activity of the first enzyme and the quantity of the second product domain provides a measurement of the activity of the second enzyme.

72-74. (canceled)

75. A method of screening compounds to identify a drug candidate comprising
measuring the activity of at least one enzyme according to the method of claim 1, in the presence and absence of at least one test compound; and
comparing the activity of the at least one enzyme in the presence and absence of the at least one test compound, wherein the method identifies an inhibitor or agonist drug candidate from reduced or increased activity, respectively, of the at least one enzyme in the presence of the at least one test compound.

76-80. (canceled)

81. A method for screening an organism for a disease, disorder, or abnormality characterized by aberrant enzymatic activity, comprising:
quantitatively measuring the activity of at least one enzyme from a cell lysate from at least one cell of the organism, according to the method of claim 1; and
comparing the measurement to a reference measurement of the activity of the at least one enzyme, wherein the presence or absence of the abnormality is identified from the comparison.

82. A method of characterizing a disease, disorder, or abnormality comprising:
quantitatively measuring the activity of at least one enzyme from a sample according to the method of claim 1, wherein the sample comprises at least one diseased cell isolated from an organism, or comprises a lysate of the at least one cell;
comparing the measurement(s) to a reference measurement of the activity of the at least one enzyme;
characterizing the disease or disorder by identifying an enzyme with elevated activity or decreased activity in the at least one cell known or suspected of being diseased compared to activity of the enzyme in non-diseased cells of the same type as the diseased cell.

83. The method of claim 82, wherein the disease, disorder, or abnormality is a neoplastic disease, inflammation, neurodegeneration, diabetes, a metabolic disorder, a cardiovascular disorder, an allergy, a neurological disorder, a nephropathy, a liver disorder, an immunological disorder, or is associated with transplant rejection.

84. (canceled)

85. The method of claim 82, further comprising administering to the organism a compound or composition that inhibits or promotes the activity of the enzyme identified as having the elevated activity or decreased activity, respectively, in the at least one diseased cell.

86. The method of claim 82, wherein the cell or cell lysate is obtained from cells from a medical biopsy obtained from the organism and snap frozen to preserve enzymatic activity.

87-93. (canceled)
